(19) United States
(12) Patent Application Publication
(10) Pub. No.: US 2023/0355413 A1
(43) Pub. Date: Nov. 9, 2023

(54) SYSTEMS AND METHODS FOR TREATING VASCULAR DISEASE

(71) Applicant: Route 92 Medical, Inc., San Mateo, CA (US)

(72) Inventors: Michi E. Garrison, San Mateo, CA (US); Craig Bonsignore, San Mateo, CA (US); John Miller, San Mateo, CA (US); Tony M. Chou, San Mateo, CA (US); Travis Carbonneau, San Mateo, CA (US)

(21) Appl. No.: 18/311,802

(22) Filed: May 3, 2023

Related U.S. Application Data
(60) Provisional application No. 63/422,762, filed on Nov. 4, 2022.
(60) Provisional application No. 63/346,524, filed on May 27, 2022.
(60) Provisional application No. 63/338,114, filed on May 4, 2022.

(51) Int. Cl. A61F 2/915

(52) U.S. Cl. CPC A61F 2/915; A61F 2002/91575; A61F 2250/0098; A61F 2230/0054

(57) ABSTRACT

A flow diverter including a self-expanding tubular member having a plurality of expandable cells, each of the expandable cells having interconnected struts and bridges. The tubular member has a constrained configuration having a first outer diameter of at least 1.0 mm sized for delivery using a flow diverter delivery system and an expanded configuration having a second outer diameter larger than the first outer diameter. The tubular member has a proximal end zone, a distal end zone, and a middle zone located between the proximal end zone and the distal end zone. At least the middle zone of the tubular member is laser-cut to have a material coverage of at least 25% when the tubular member is in the expanded configuration. Related devices, systems, and methods of treating disease, particularly intracranial and cerebral aneurysms by deploying implantable expandable devices, are provided.

### CROSS-REFERENCE TO RELATED APPLICATIONS

This application claims the benefit of priority under 35 U.S.C. § 119(e) to U.S. Provisional Patent Application Serial Nos. 63/338,114, filed May 4, 2022, 63/346,524, filed May 27, 2022, and 63/422,762, filed Nov. 4, 2022. The disclosures are hereby incorporated by reference in their entireties.

### FIELD

The present technology relates generally to medical device systems and methods, and more particularly, to medical device delivery and methods of implantation of stents or flow diverters for the treatment of vascular disease, such as intracranial aneurysm.

### BACKGROUND

Endovascular implantation of scaffolding devices, such as flow diverters, are used to treat aneurysms in vessels of the brain (e.g., cerebral arteries) or vessels leading to the brain (e.g., intracranial arteries). Flow diverters are particularly useful for treating aneurysms with wide necks that are difficult to exclude by other means, such as embolic coils. The flow diverters are targeted to be positioned starting from a distal normal segment, spanning the aneurysm, and ending in a proximal normal segment. Flow diverters are designed to have a very dense material coverage, around 30% when expanded, so as to exclude or limit blood flow from entering the aneurysm through the aneurysm neck. Excluding blood flow into the aneurysm reduces or eliminates the risk of aneurysm rupture due to thrombosis at the site over time.

In general, endovascular implantation of devices in cerebral and intracranial arteries have been performed via smaller-sized delivery systems. Access with larger diameter systems has been challenging due, in part, to the tortuosity of the vasculature in the skull as well as the small size and delicate nature of the vessels. Navigating these arteries to deliver endovascular implants such as flow diverters requires catheter systems having superior flexibility and deliverability, which can be challenging, especially for larger diameter catheters. Due to difficulty in navigating large-diameter delivery systems to these anatomies, flow diverters and other endovascular implants have been typically delivered through microcatheters that have an inner diameter of 0.027″ or smaller. Furthermore, the implants are typically pushed through these catheters rather than being delivered pre-mounted on the distal end of a delivery system, as is standard for self-expanding stents in larger and more accessible locations. This method requires extra steps and wire exchanges, adding to the time and risk of the procedure.

All currently available flow diverters are based on a braided wire design to achieve the high percentage metal coverage that achieves the desired thrombotic effect. A braid design can expand from a diameter deliverable through a 0.027″ inner diameter microcatheter to a vessel having a maximum desired vessel diameter of up to 5 mm (0.2″) while still possessing a metal coverage ratio of 30% at the expanded configuration. Examples include the Medtronic PIPELINE, the Stryker SURPASS, the Terumo FRED, and others. In contrast, stents constructed from laser-cut metal tubes, such as Nitinol, stainless steel, and other alloys are unable to accomplish the desired at least 30% metal coverage ratio due to geometric constraints.

Unfortunately, braid-style flow diverters can be difficult, time-consuming, imprecise, and risky to deliver. One problem with braid-style, self-expanding implants, such as braided flow diverters is that they may not immediately expand fully to the walls of the vessel and therefore may move during deployment, leading to time-consuming and risky maneuvers to achieve the desired wall coverage, location, and wall apposition. Significant shortening of the braided flow diverters is also a problem during deployment due to the nature of braid construction, and often leads to ineffective coverage of the aneurysm site and often requires repositioning, manipulation, or may require placement of an additional implant. Because of this, coverage of the aneurysm and/or apposition of the flow diverter against the wall is often not optimal. Poor apposition is associated with higher rates of narrowing or occlusion of the flow diverter.

Additionally, due to difficulty in navigating large diameter delivery systems to distal carotid and cerebral anatomies, devices such as flow diverters have been typically delivered through microcatheters that are 0.027″ ID or smaller. The delivery system for such devices often includes a leading distal guidewire tip, which presents risk of vessel perforation. Furthermore, braid-style implants like flow diverters terminating in wire ends often require delivery systems with additional distal-end-constraining features to enable the device to be pushed through the microcatheter. This constraining feature adds time and complexity to the deployment procedure.

There is a need for improved flow diverters that can be delivered through larger-bore access systems that are able to optimally access cerebral and intracranial arteries for the treatment of aneurysms at these sites while providing adequate vessel coverage and improved deliverability and expansion characteristics. There is also a need for improved flow diverter delivery systems of flow diverters which are compatible with these larger-bore access devices and which can deliver flow diverters precisely and quickly with minimal steps.

### SUMMARY

In an aspect, provided is a flow diverter including a self-expanding tubular member having a plurality of expandable cells, each of the expandable cells having interconnected struts and bridges. The tubular member has a constrained configuration having a first outer diameter of at least 1.0 mm sized for delivery using a flow diverter delivery system and an expanded configuration having a second outer diameter larger than the first outer diameter. The tubular member has a proximal end zone, a distal end zone, and a middle zone located between the proximal end zone and the distal end zone. At least the middle zone of the tubular member is laser-cut to have a material coverage of at least 25% when the tubular member is in the expanded configuration.

The interconnected struts and bridges of each expandable cell can include two pairs of struts each strut of the two pairs of struts having an outer edge. The outer edge of a first strut of a first pair can be interconnected to an outer edge of a second strut of the first pair by one of the bridges. The first strut of the first pair of struts can connect at a central bend to a first strut of a second pair of struts and the second strut of the first pair can connect at a central bend to a second strut of the second pair. A circumferential height from the central bend of the first pair to the outer edge of the first pair is Y and an axial distance from the central bend of the first pair to the outer edge of the first pair is X, and wherein a diagonal of a rectangle defined by X and Y can be equal to a length of the first strut. A ratio of the length of the first strut to the circumferential height of the first strut can be between 1 and 5. Each of the pairs of struts can be arranged parallel to one another and spaced an axial distance away from one another thereby defining a V-shaped opening of the expandable cell. The two pairs of struts can be interconnected to form a peak on a first end of the expandable cell and a corresponding valley on a second end of the expandable cell. The plurality of expandable cells can be arranged in circumferential rings and each peak in a circumferential ring of expandable cells can be aligned circumferentially with each peak of an adjacent circumferential ring of expandable cells. A bridge can connect the peak of the expandable cell of a first circumferential ring to a valley of an expandable cell of an adjacent second circumferential ring.

The middle zone can have properties different from one or both of the proximal end zone and distal end zone. The middle zone can have greater material coverage than one or both of the proximal end zone and the distal end zone. One or both of the proximal end zone and the distal end zone can be laser-cut to have a material coverage that is less than the material coverage of the middle zone. The material coverage of the middle zone can be between 25%-35% when the tubular member is in the expanded configuration and the proximal and distal end zones can have a material coverage less than the material coverage of the middle zone. At least one of the proximal end zone, the middle zone, and the distal end zone can include at least one radiopaque marker. A length of the flow diverter in the constrained configuration can be less than 1% different from a length of the flow diverter in the expanded configuration. A length of the flow diverter in the constrained configuration can be less than about 5% different from a length of the flow diverter in the expanded configuration. A length of the flow diverter in the constrained configuration can be less than about 10% different from a length of the flow diverter in the expanded configuration.

The first outer diameter can be between 1.5 mm and 2.5 mm and wherein the second outer diameter is between 2.0 mm and 6.0 mm. A length of the flow diverter in the constrained configuration can be between 10 mm and 35 mm. The plurality of expandable cells of the tubular member can be arranged into between 10 and 50 circumferential rings. A pitch of the middle zone can be between about 0.25 mm-0.40 mm, the pitch corresponding to a length of a bridge of an expandable cell of the middle zone. A pitch of one or both of the proximal end zone and distal end zone can be about 0.45 mm-0.75 mm, the pitch corresponding to a length of a bridge of an expandable cell of the proximal end zone or distal end zone. The plurality of expandable cells can form rows extending between proximal and distal ends of the tubular member parallel with a longitudinal axis of the tubular member, the rows of the expandable cells aligned peak-to-valley. The tubular member can include between 4 and 10 rows.

At least the distal end zone can include a rail formed of bridges interconnecting the plurality of expandable cells within a row. The rail can enable resheathing of the distal end zone in a delivery system after at least partial deployment of the distal end zone. One or both of the proximal end zone and the distal end zone can include a braided or woven construction. The interconnected struts can be connected by hinges in a plurality of V-shapes. A line connecting radially adjacent hinges can pass through at least 4 cells in the middle zone. The line connecting radially adjacent hinges in the proximal and distal zones can pass through fewer cells than in the middle zone. Bridges located in the middle zone can be shorter than bridges located in the distal end zone and proximal end zone. The struts in the middle zone, distal end zone, and proximal end zone can be substantially the same in length and configuration. The bridges can lie parallel to a flow diverter central axis.

In an interrelated aspect, provided is a method of treating intracranial or cerebral aneurysm including advancing a catheter system through a base sheath towards an intracranial or cerebral vessel having a segment with an aneurysm. The catheter system includes an inner catheter having a tubular elongate body with a single lumen and a flexible, distal tapered end region; and an outer catheter having a catheter lumen and a distal end. The method includes positioning the tapered end region of the inner catheter distal to the distal end of the outer catheter; crossing the segment of vessel with the aneurysm with at least a portion of the tapered end region of the inner catheter; advancing the outer catheter over the inner catheter and positioning a distal end region of the outer catheter across the lesion; withdrawing the inner catheter from the catheter lumen and maintaining the outer catheter in place across the aneurysm; advancing a flow diverter delivery system comprising a flow diverter through the catheter lumen to the distal end region of the outer catheter; withdrawing the outer catheter while maintaining the flow diverter delivery system in place; and deploying the flow diverter across the segment with the aneurysm.

In an interrelated aspect, provided is a method of performing a medical procedure at a treatment site in a brain of a patient including positioning a system of devices into an advancement configuration. The system of devices includes a catheter having a catheter lumen, an inner diameter, and a distal end; and an inner member sized and shaped to slide within the catheter lumen. The inner member defines a single lumen and has a distal portion. The distal portion has a first outer diameter that tapers distally to a second outer diameter that is smaller than the first outer diameter, and the inner member transitions in flexibility from a proximal end of the inner member to a distal end of the inner member, the distal end of the inner member being more flexible than the distal end of the catheter. When positioned in an advancement configuration, the inner member extends coaxially through the catheter lumen until the distal portion of the inner member is positioned distal to the distal end of the catheter. The method includes advancing the catheter and the flexible inner member to a target location to an access point of entry while the system of devices is positioned in the advancement configuration; positioning the catheter at the treatment site, the treatment site comprising an aneurysm; removing the inner member from the patient; and treating the aneurysm through the catheter. The step of treating can include delivering a flow diverter to the aneurysm through the catheter.

In an interrelated aspect, provided is a flow diverter delivery system including a flow diverter having a tubular structure and configured to treat an aneurysm in an intracranial vessel, the flow diverter having a constrained configuration having a first outer diameter and an expanded configuration having a second outer diameter; an inner core member including an elongate shaft having a recessed region near a distal end region of the elongate shaft, the recessed region sized to receive the tubular structure of the flow diverter when the flow diverter is in the constrained configuration; and an atraumatic distal tip region located distal to the recessed region. The distal tip region has a taper from a first outer diameter of the elongate shaft to a second outer diameter of the elongate shaft. The first outer diameter of the elongate shaft is larger than an outer diameter of the recessed region. The system includes an outer restraining sleeve having an inner diameter sized to receive the inner core member and the flow diverter in the constrained configuration. The outer restraining sleeve is retractable at least a distance to deploy the flow diverter.

The inner diameter of the restraining sleeve can be size-matched to the first outer diameter of the elongate shaft to reduce an annular space at a leading end of the flow diverter delivery system. The distal tip region can include at least one radiopaque marker at a distal end. The distal tip region can include a second radiopaque marker positioned to identify the taper.

In an interrelated aspect, provided is a flow diverter having a self-expanding tubular member having a proximal end, a distal end, and a longitudinal axis. The tubular member has a constrained configuration with a first outer diameter sized for delivery and an expanded configuration having a second outer diameter larger than the first outer diameter. The tubular member includes a plurality of expandable cells, each cell having interconnected struts and bridges arranged in circumferential rings. The circumferential rings form rows of the expandable cells extending between the proximal and distal ends of the tubular member parallel with the longitudinal axis, the rows of expandable cells nested peak-to-valley. The tubular member has a proximal end zone near the proximal end of the tubular member, a distal end zone near the distal end of the tubular member, and a middle zone located between the proximal end zone and the distal end zone. At least the distal end zone includes at least one rail formed of bridges interconnecting each circumferential ring of expandable cells within a single row.

The at least one rail can enable resheathing of the distal end zone in a delivery system after at least partial deployment of the distal end zone from the delivery system. At least the middle zone of the tubular member can be laser-cut to have a material coverage of at least 25% when the tubular member is in the expanded configuration.

In an interrelated aspect, provided is a flow diverter configured to expand from a constrained state to an expanded state. The flow diverter includes a first tube of superelastic material formed of a plurality of cells having a first material coverage; and a second tube of superelastic material formed of a plurality of cells having a second material coverage. The second tube is positioned inside of the first tube so that an overlap of the plurality of expandable cells of the first tube and the plurality of expandable cells of the second tube forms a third material coverage that is greater than the first material coverage and the second material coverage when the flow diverter is in the expanded state.

The second tube can be locked in position inside the first tube by a feature in a cut pattern of at least one of the first tube and the second tube. The feature can include a slot in the first or the second tube and tab configured to protrude into the slot to lock the first and second tubes together. The feature can include a hole in the first or the second tube and a malleable disk configured to insert within the hole to lock the first and second tubes together. At least one of the first tube and the second tube can be non-braided and laser-cut.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A is an implementation of a cut-tube flow diverter in the collapsed delivery configuration;

FIG. 1B is the flow diverter of FIG. 1A in the expanded configuration;

FIG. 1C is the flow diverter of FIG. 1B in the expanded configuration showing a flat (unrolled) view of the device pattern;

FIG. 1D is an interrelated implementation of a cut-tube flow diverter;

FIG. 1E is the flow diverter of FIG. 1D showing a flat (unrolled) view of the device pattern;

FIG. 1F is the flow diverter of FIG. 1D in an as-cut configuration showing details of the cut pattern;

FIG. 2A shows a cell of a section of the flow diverter of FIG. 1C;

FIGS. 2B-2D show cells of different sections of the flow diverter of FIG. 1C having different pitches;

FIG. 3 shows additional details of an implementation of a cut-tube flow diverter;

FIGS. 4A-4C show details of an attachment mechanism between two layers of a dual-layer flow diverter;

FIGS. 5A-5B show details of an alternate attachment mechanism between two layers of a dual-layer flow diverter;

FIGS. 6A-6B show implementations of a dual layer flow diverter;

FIG. 7 shows an implementation of a compound flow diverter;

FIG. 8A shows components of a flow diverter and delivery system for the flow diverter;

FIG. 8B shows the flow diverter and delivery system of FIG. 8A assembled in a delivery configuration;

FIG. 8C shows the flow diverter and delivery system of FIG. 8B with the flow diverter partially deployed;

FIG. 9A shows an access catheter system for accessing a treatment site in an artery;

FIG. 9B shows the access catheter system of FIG. 9A assembled for use;

FIG. 10A is a detail view of a distal end region of a catheter advancement element taken along circle C-C of FIG. 9A;

FIG. 10B is a detail view of a distal end region of a catheter advancing element having a guidewire positioned in the inner lumen of the catheter advancement element of FIG. 10A;

FIG. 11A shows an assembled catheter system accessing an intracranial aneurysm, with a base sheath positioned in the internal carotid artery (ICA), an outer catheter advanced in the distal ICA, and an inner catheter crossing the vessel in the area of the aneurysm;

FIG. 11B shows the outer catheter of FIG. 11A advanced across the aneurysm and the inner catheter withdrawn;

FIG. 11C shows show a flow diverter delivery system advanced across the aneurysm and the outer catheter withdrawn;

FIG. 11D shows the flow diverter restraining sleeve withdrawn and a flow diverter deployed across the aneurysm;

FIG. 12A is a flat (unrolled) view of an interrelated implementation of a cut-tube flow diverter in the collapsed state;

FIG. 12B shows a detailed section of the flow diverter of FIG. 12A taken at circle A;

FIG. 13 is a flat (unrolled) view of an interrelated implementation of a cut-tube flow diverter in the collapsed state;

FIG. 14 is an interrelated implementation of a cut-tube flow diverter with flared ends.

It should be appreciated that the drawings are for example only and are not meant to be to scale. The drawings are intended to be illustrative to dimensions including metal coverage percentages. The drawings are not to scale in absolute terms or comparatively. It is to be understood that devices described herein may include features not necessarily depicted in each figure.

### DETAILED DESCRIPTION

Described herein are flow diverters and delivery systems and methods that are compatible with large-bore access systems. The devices, systems and methods take advantage of the large-bore access to provide improvements over existing braided flow diverters and associated microcatheter-based delivery systems to enable more precise, safe, and rapid treatment of aneurysms. These devices and systems can be delivered through any large-bore neurovascular access systems. Also described are improved large-bore access systems that facilitate the speed, safety, and ease of accessing intracranial and cerebral arteries to implant flow diverters at their intended site, even despite navigational challenges.

Where the phrase “access catheter” is used herein that such a catheter may be used for other purposes besides or in addition to access, such as the delivery of fluids to a treatment site or as an aspiration catheter. Alternatively, the access systems described herein may also be useful for access to other parts of the body outside the vasculature. Similarly, where the working device is described as being an expandable cerebral treatment device or flow diverter, other interventional devices can be delivered using the access and delivery systems described herein. As used herein, “an aneurysm” refers to the ballooning out of a weakened section of vessel wall. A “cerebral aneurysm” or “intracranial aneurysm” refers to an aneurysm in a vessel of the brain.

### Flow Diverters

Disclosed herein are flow diverters that greatly improve the deployment and performance compared to current braided-style flow diverters. The flow diverters can be self-expanding, cut-tube style implants that unlike a braided wire tube, expand to full diameter much more quickly and accurately. There is no need to constrain or cover the distal end of the implant because the cut-tube construction lacks wire ends like braided implants do. The cut-tube flow diverters described herein also do not experience significant foreshortening as braided wire scaffolds do. The cut pattern can be designed to achieve wall apposition and coverage sufficient to achieve flow diversion, prevention, or rejection of blood flow into the aneurysm, and/or isolation of an aneurysm, with a single layer of material (as opposed to a braid, with a crossed wire surface), resulting in a smoother and less thrombogenic inner surface.

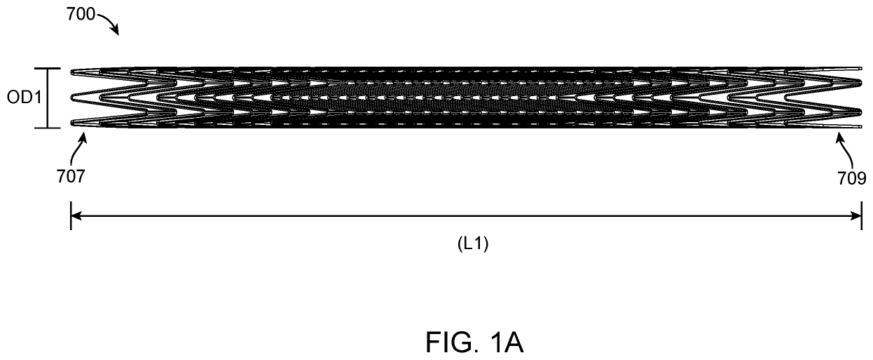

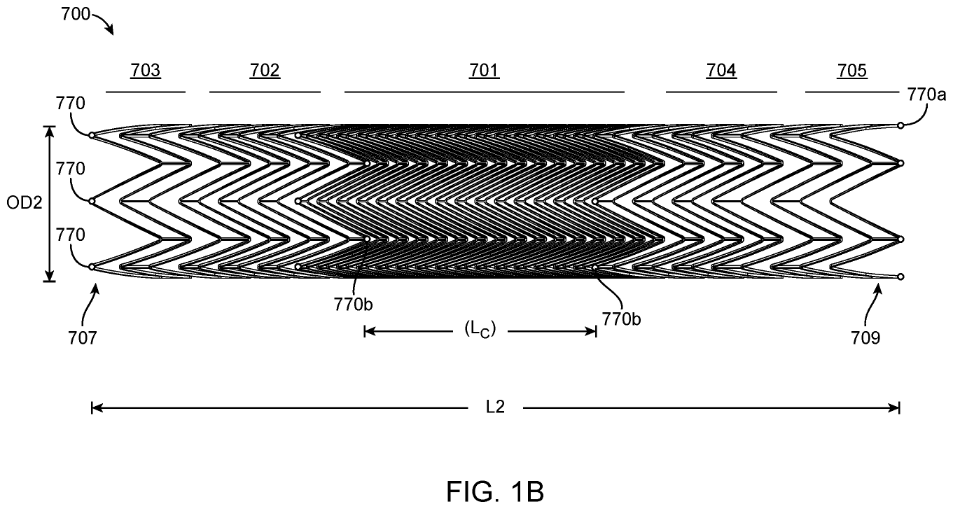

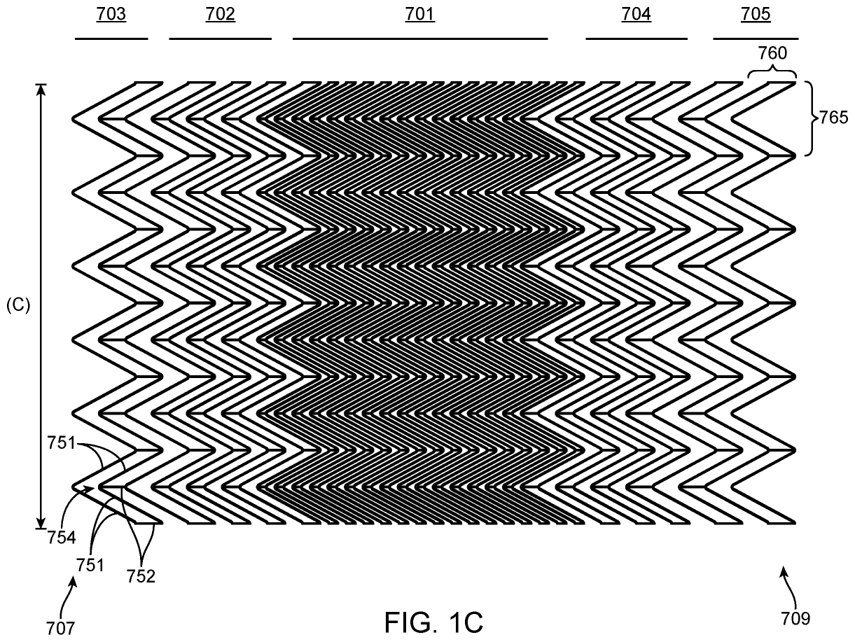

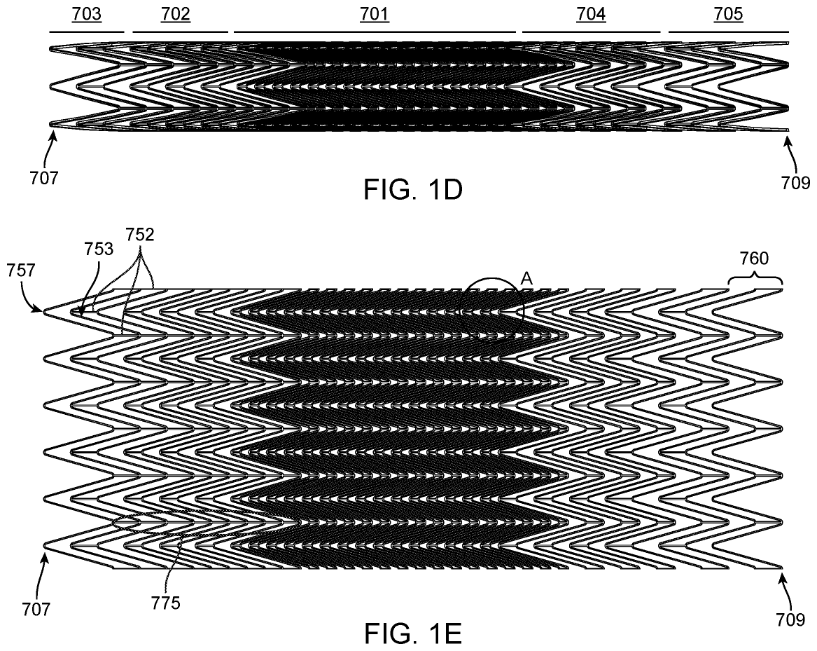

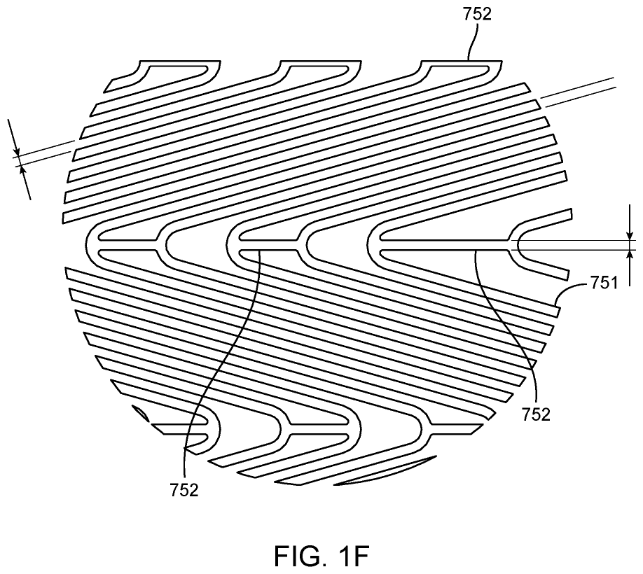

FIGS. 1A-1C and FIGS. 1D-1F show implementations of cut-tube-style flow diverter 700. In each implementation, the flow diverter 700 is a generally tubular member or element that is non-braided and having an open distal end 707 and an open proximal end 709, the distal end 707 being further away from the user during advancement through a vessel and the proximal end 709 being closer to the user during advancement through the vessel. A longitudinal axis extends between the distal and proximal ends 707, 709. FIG. 1A shows the flow diverter 700 in a constrained configuration having a first outer diameter OD1 and first length L1. The first outer diameter OD1 accommodates insertion of the flow diverter 700 into and navigation through the vasculature to the treatment site. FIG. 1B shows the flow diverter 700 in the expanded configuration having second outer diameter OD2 and a second length L2. Upon reaching the treatment site, the flow diverter 700 is deployed and expands to the second outer diameter OD2. Thus, OD2 is larger than OD1.

The constrained outer diameter OD1 of the flow diverter 700 can be about 0.89 mm (0.035″) to about 3 mm (0.118″), preferably about 1.5 mm (0.06″) to 2.5 mm (0.10″), or about 1.60 mm. This constrained outer diameter OD1 is relatively large compared to the constrained outer diameter of a conventional braided-style flow diverters. This is enabled by the larger-bore access system described herein that is configured to reach distal sites for deployment of the flow diverter. The deliverability of the large-bore access system, which will be described in more detail below, is capable of delivering the larger constrained outer diameter OD1 flow diverter, also enables the flow diverter to be designed with greater material coverage when expanded.

The expanded outer diameter OD2 of the flow diverter 700 can be about 2 mm (0.08″) up to about 6 mm (0.24″), preferably between about 2.5 mm (0.10″) and about 5 mm (0.2″), depending on the anatomic requirements. The flow diverter 700, when expanded, is preferably suitable for vessels up to 5 mm in diameter. The flow diverter length L1 can also be manufactured depending on the anatomic requirements. For example, the length L1 of the flow diverter prior to expansion can vary from 10 mm to 50 mm including any dimension between and preferably from 10 mm to 35 mm.

The length L2 of the flow diverter after expansion can be in the same range. The cut-tube-style flow diverters 700 described herein undergo a minimal amount of foreshortening upon deployment from the constrained state to the expanded state in the vessel. This provides an advantage over braided-wire style flow diverters that significantly foreshorten upon deployment. The flow diverter designs shown in FIGS. 1A-1C and also FIG. 1D-1F shorten less than 10%, less than 5%, or about 1% or less when expanded from about 1.6 mm to about 4.0 mm. For example, the constrained length L1 can be about 20.7 mm and the expanded length L2 can be about 20.5 mm, which is less than about 1% foreshortening. In contrast, a braided stent shortens by about 50% when expanded to 4 mm (see Instructions For Use of the Surpass Evolve Flow Diverter System; Stryker Neurovascular). This extreme foreshortening of braided flow diverters requires precision placement of the distal end to ensure full coverage of the target segment. The distal end must extend from a site distal to the aneurysm in a normal vessel segment so that the middle section of the flow diverter can be positioned across the aneurysm neck and to a normal vessel on the proximal side of the aneurysm. Extreme foreshortening of a flow diverter can lead to “missed” deployments especially in curved or tortuous anatomy. If the aneurysm neck is very large or if the vessel is tapered at the aneurysm neck, the final length and position is even more unpredictable.

The flow diverter 700, when the tubular member is in the expanded, is capable of achieving a material coverage suitable for treating aneurysms. The material coverage (also referred to herein as material density) can vary, but is generally between about 25%-35% material coverage (+/−5%). “Material coverage” as used herein means the surface area of the outer surface of the flow diverter divided by the surface area of the inner lumen of the vessel. The material of the flow diverter provides the material coverage, which is the inverse of porosity of the flow diverter, which is a function of the amount of open space of the flow diverter upon expansion.

FIGS. 1C and 1E are unrolled views showing the flat pattern of the flow diverters. The flow diverter 700 includes a plurality of repeating cells 750 arranged in a plurality of circumferential rings 760 that repeat along an axial length of the flow diverter 700 between the proximal end 709 and distal end 707. The cells 750 are radially and circumferentially aligned with adjacent cells 750 forming a repeating zig-zag pattern. The cells 750 aligned axially so that they nest with one another creating a plurality of axial rows 765. The number of axial rows 765 is thus controlled by the number of cells 750 around the circumference of the flow diverter within a single ring 760.

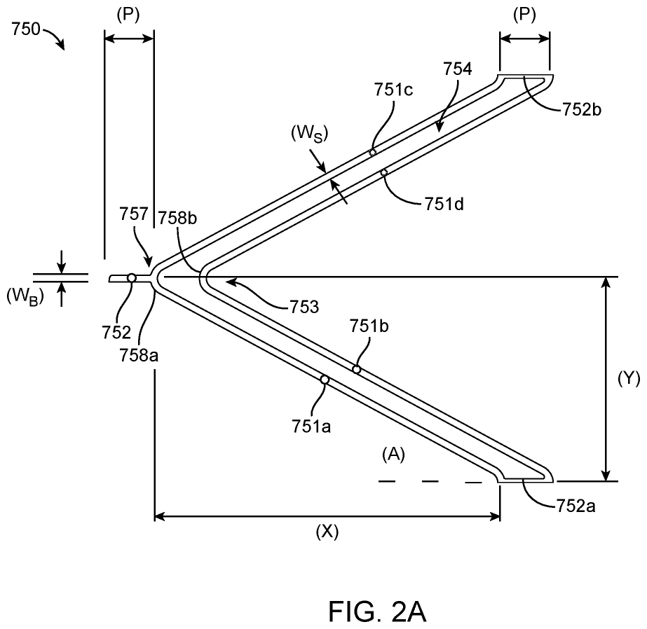

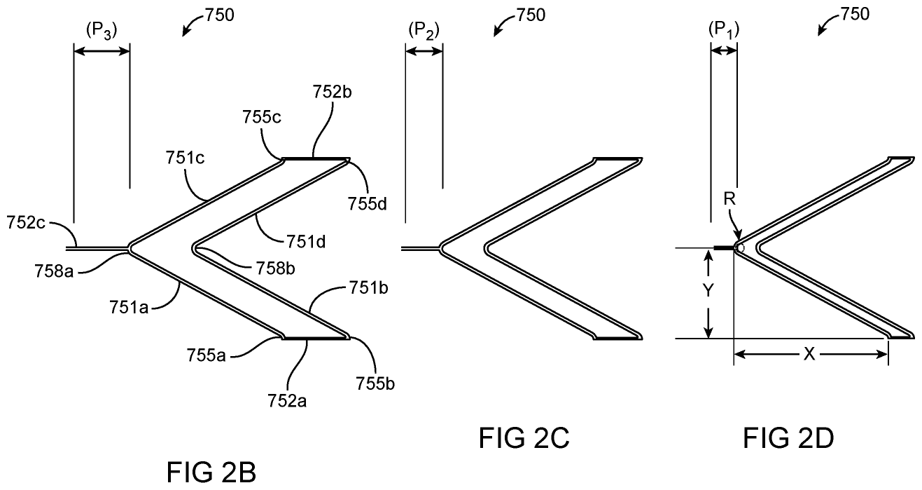

The flow diverter is self-expanding and can be a tubular member having circumferential rings of expandable cells 750. Each of the cells 750 includes interconnected struts and axial bridges. FIG. 2A shows a single cell 750 of the flow diverter 700 of FIG. 1A and FIG. 1D including an arrangement of interconnected struts 751 and axial bridges 752. Each cell 750 can be formed by four struts 751 with a first two of the four struts 751 coupled at a first central bend or hinge into a V-shape and a second two of the four struts 751 connected at a second central bend or hinge into a V-shape. The central bends (also referred to herein as hinges) are aligned and spaced a distance apart so that the pairs of struts 751 are spaced a distance away from one another. Said in another way, each cell incorporates two V-shaped struts 751, each including a central bend and two straight arms that project in opposite directions at an angle away from the central bend. The struts 751 in each cell 750 are arranged so that the central bends nest a distance apart. The angle of the central bends can be equal so struts 751 are positioned parallel to one another thereby defining a V-shaped opening 754 for each cell 750. Regardless whether the cells 750 are considered to have two V-shaped struts or four struts arranged into two Vs, the plurality of struts 751 form a zig-zag pattern around the circumference of the flow diverter 700. Additionally, the zig-zag pattern of struts 751 need not be limited to V-shapes and can include “W-shape,” “Z-shape,” or “M-shape” strut patterns as well as other strut patterns such as diamond shapes or patterns forming an open-cell or closed-cell structure.

Each expandable cell 750 with the V-shaped opening 754 forms a valley 753 opening towards the proximal end 709 of the flow diverter 700 and a peak 757 projecting towards the distal end 707 of the flow diverter. The peak 757 is formed by the central bend 758 between two struts 751 and the valley 753 is formed by the central bend 758 of the other two struts 751. The first pair of struts 751 can be connected to one another at a first edge of the cell 750 by a first bridge 752 and the second pair of struts 751 can be connected to one another at the opposite edge of the cell 750 by a second bridge 752. A third bridge 752 can couple the peak 757 of one cell 750 to the valley 753 of an adjacent cell 750, which will be described in more detail below.

The V-shaped openings 754 formed by the struts 751 and connecting bridges 752 can repeat around a circumference of the flow diverter 700 forming one of the plurality of circumferential rings 760. The pattern of cells 750 can repeat along the axial length of the flow diverter 700 a number of times thereby defining the number of rings 760. Depending on the desired overall axial length and density of cells, the flow diverter 700 can include between 10 and 50 rings 760, and any number in between, of zig-zagging V-shaped cells 750 depending on the overall length of the flow diverter 700 and the needs of the anatomy being treated. The pattern of cells 750 can repeat circumferentially around the tubular structure of the flow diverter 700 a number of times thereby defining the number of rows 765. The number of rows 765 may vary depending on the desired dimensions of the flow diverter 700. For the dimensions typical for intracranial and cerebral aneurysm treatment, the number of rows 765 may range from 4 to 10, preferably about 6. Thus, the number of struts 751 around the circumference of the flow diverter, which is twice the number of peaks 753 because four struts 751 pair to create a single cell 750 with a peak 753, can be about 8 to 20, preferably about 12. FIGS. 1C and 1E show a row 765 including cells 750 with their peaks 757 pointing toward the distal end 707 of the flow diverter 700 and their valleys 753 opening toward the proximal end 709 of the flow diverter 700. With this arrangement, a single ring 760 includes 6 rows 765 of full cells 750.

Still with respect to FIG. 1C and also FIG. 1E, the rings 760 of cells 750 can be connected to neighboring rings 760 via a bridge 752. The bridge 752 connects the peak 757 of a first cell 750 of a first ring 760 connects to a valley 753 of an adjacent cell 750 of a second ring 760. The peak-to-valley connections allow for each ring 760 of V-shaped cells 750 to tightly nest with one another when in the collapsed configuration and also maintain a high material coverage when deployed to the expanded configuration. The tight nesting and high material coverage upon deployment is also a function of the long struts 751 and the relatively short bridges 752 that connect them peak-to-valley.

The axial length of these bridges 752 controls the axial spacing between the struts 751 in the cell 750, which is also referred to herein as the pitch P (see FIG. 2A). This axial length and axial spacing is one factor controlling the material coverage of the flow diverter and the porosity of a flow diverter, which is the inverse of the material coverage. The material of the struts 751 provides the material coverage and the openings 754 between the struts 751 and openings 756 between the cells 750 creates open space of the flow diverter and is the porosity.

FIGS. 2A-2D show single cells 750 formed of interconnected struts 751 and axial bridges 752. As mentioned above, each of the cells 750 can be formed by four struts 751. The four struts 751 can be grouped into pairs. Each strut of the two pairs of struts can include an outer edge 755. The outer edge 755a of a first strut 751a of a first pair is interconnected to a corresponding outer edge 755b of a second strut 751b of the first pair by one of the axial bridges 752a. The first strut 751a of the first pair of struts connects at a central bend 758a to a first strut 751c of a second pair of struts. The second strut 751b of the first pair connects at a central bend 758b to a second strut 751d of the second pair of struts. The outer edge 755c of the first strut 751c of the second pair of struts is interconnected to a corresponding outer edge 755d of a second strut 751d of the second pair by another axial bridge 752b. A third bridge 752c projects distally from the central bend 758a.

Still with respect to FIGS. 2A-2D, Y is the circumferential height occupied by a single strut 751 from the central bend 758 near the peak 757 of the cell 750 to the outer edge 755 of the cell 750. X is the axial distance occupied by a single strut 751 from the central bend 758 near the peak 757 of the cell 750 to the outer edge 755 of the cell 750. Strut length is approximately equal to the diagonal of a rectangle defined by X and Y. The ratio of the strut length to the circumferential height Y of a strut 751 in the expanded configuration controls the angle of the struts 751 projecting away from the central bend 758 near the peak 757. This ratio can be from 1 to 5, 1 to 3, preferably around 2. The ratio of the radius R of each central bend 758 (see FIG. 2D) to the circumferential height of a strut Y in the constrained configuration controls the radius of curvature of each peak 757. The ratio can be in the range 2 to 10, preferably around 6. Strut width is shown in FIG. 2A as W_{S} and bridge width is shown as W_{S}. The strut axial and circumferential height X and Y, strut width W_{S}, axial bridge length P and bridge width W_{S}, are selected to meet certain criteria.

The bridges 752 are shown as connecting members that are substantially straight and lie parallel to a longitudinal axis of the flow diverter (i.e., central axis). The bridges 752 connect adjacent peaks and valleys to form a cell 750. However, the bridges 752 need not be straight and can also be curved or angled relative to the longitudinal axis of the flow diverter. The bridges 752 also can connect at a location other than a peak to a valley such as connected at a middle of a strut 751 or at a point where a strut 751 meets a hinge (i.e., central bend 758). Further, the bridges 752 are shown aligned and connecting every 2^{nd} pair of struts in a longitudinal direction. However, the bridges need not be axially aligned at all or may be axially aligned at every 3rd, 4th, 5th or other number of strut pairs or cells. Use of the term “axial” herein means in a direction that is along a length of the tubular member of the flow diverter, but does not require the direction to be parallel to the longitudinal axis of the flow diverter. For example, “axial bridge” includes bridges that lie parallel to the central axis of the flow diverter and also bridges that are angled, stepped, or curved relative to the central axis of the flow diverter.

When in the collapsed configuration as shown in FIG. 1A, the struts 751 of each cell 750 are close to touching or touching in at least a region of each cell 750. The spacing of the struts 751 can be selected so that the struts 751 do not overlap one another at the constrained diameter of OD1. The area of the V-shaped opening 754 between the struts 751 of each cell 750 can be diminished to nearly zero so that only the area of the V-shaped opening 754 near the central bends of the strut pair or between the valleys 753 and the peaks 757 of each cell 750 remains. When in the expanded configuration as shown in FIG. 1B, the struts 751 of each cell 750 may expand away from one another so as to no longer touch. FIG. 1F is a detail view of FIG. 1E taken at circle A. Each strut 751 in this region is spaced a distance away from the neighboring struts by a space that is as narrow as the strut width. This spacing between the struts 751 of the cells 750 provides the high material coverage of the flow diverter when in the expanded configuration. The flow diverter 700 preferably has a material coverage of between 25%-35% when in the expanded diameter of OD2.

Table 1 below provides example parameters for the flow diverter 700 shown in FIGS. 1A-1C including the parameters such as pitch P1 of the central portion of the flow diverter. The material coverage in the expanded configuration can be between about 25% and about 35%. The outer diameter in the collapsed configuration OD1 can be between about 1.5 mm and about 2.5 mm. The outer diameter of the flow diverter in the expanded configuration OD2 can be between about 2 mm and about 6 mm. The length of the flow diverter in the collapsed configuration L1 and in the expanded configuration L2 can be between about 10 and about 50 mm. The change in length between the collapsed configuration and the expanded configuration can be less than 10%, less than 5%, and less than 1%. The number of rows 765 in the flow diverter can be between about 4 and about 10 and the number or rings 760 can be between about 10 and about 30. The pitch P of each cell 750 in the densest part of the flow diverter can be between about 0.25 mm and about 0.75 mm depending on the material coverage desired. The circumferential height Y of a single strut can be between about 0.5 mm and about 1.5 mm and the axial distance X can be between about 1.0 mm and about 2.0 mm. The bridge width W_{S} can be between about 0.025 mm and about 0.09 mm. The strut width W_{S} can be between about 0.025 mm and about 0.09 mm.

TABLE 1

| Material | | | | | | | | | | | |
|---|---|---|---|---|---|---|---|---|---|---|---|
| coverage | OD1 | OD2 | L1 | L2 | # of | # of | P1 | Y | X | W_{B} | Wₛ |
| (%) | (mm) | (mm) | (mm) | (mm) | rows | rings | (mm) | (mm) | (mm) | (mm) | (mm) |
| 30 | 1.6 | 4.0 | 20.7 | 20.5 | 6 | 25 | 0.25 | 1.0 | 1.8 | 0.045 | 0.045 |

Other combinations of parameters may be selected to meet the criteria described above. Other features, such as strut radius, tube wall thickness, may also be selected so that the flow diverter 700 attains the desired physical properties. For example, the strut radius can be about 0.05 mm-0.10 mm, preferably about 0.07 mm and the tube wall thickness may be about 0.05 mm to about 0.09 mm, preferably between 0.065 mm-0.075 mm.

The flow diverter 700 can be designed to have a consistent pattern along its length or can vary in pattern over its length. Still further, the flow diverter 700 can have the same pattern of cells along its entire length, but the pitch of the cells changes depending on the needs of the anatomy. The differences in pattern along the length of the flow diverter 700 can be selected to modify the strength and percent material coverage for optimal performance. The variation in pattern can form different zones along the length of the flow diverter. Again with respect to FIG. 1C, rings 765 of cells 750 within a central portion of the flow diverter 700 can have a pitch P that creates a dense, middle flow diversion zone 701. The middle flow diversion zone 701 of the flow diverter 700 can have a maximum material density so as to divert flow away from anatomy external to the zone, such as an aneurysm. The flow diverter 700 can have intermediate density zones 702, 704 on either side of the middle flow diversion zone 701. The intermediate density zones 702, 704 can have a material density or material coverage that is lower than the middle zone 701. The flow diverter 700 can have two end zones 703, 705 on either side, respectively, of the intermediate density zones 702, 704, with yet lower material density than intermediate density zones 702 and 704. The material density or material coverage of the zones can be a function of the pitch P, or axial spacing between adjacent struts 751, which is a function of bridge length. At least the middle zone of the tubular member is laser-cut to have a material coverage of at least about 25% when the tubular member is in the expanded configuration. The middle zone can have different properties from one or both of the proximal end zone and the distal end zone. The middle zone can have greater material coverage than one or both of the proximal end zone and the distal end zone. One or both of the proximal end zone and the distal end zone can be laser-cut to have a material coverage that is less than the material coverage of the middle zone. One or both of the proximal end zone and the distal end zone can include a different construction compared to the middle zone, such as a braided or woven construction.

The length of the different zones can vary as well. The middle flow diversion zone 701 can have a length L_{c} designed to be longer or shorter in order to match the needs of the anatomy being treated. As an example, a flow diverter having an overall length L2 of about 20 mm, can incorporate a middle flow diversion zone 701 having a length L_{c} that is between 10 mm and 15 mm. The flow diverter can have a variety of lengths L2, expanded diameters OD2, and middle zone lengths L_{c}.

The middle flow diversion zone 701 can be formed by a plurality of rings 760 that are configured to nest tightly with one another, even after expansion, due to the relatively short length of the bridge 752 connecting the peaks 757 and valleys 753 of the cells 750 in adjacent rings 760. The length of the bridge 752 controls the pitch of the cell 750. The greater the length of the bridge 752, the greater the pitch or spacing between struts 751 of the cells 750 and the lower the material density. FIG. 2B shows a cell 750 of a distal or proximal end zone 703 or 705, with pitch P3. FIG. 2C shows a cell 750 of a transition or intermediate density zone 702 or 704, with pitch P2. FIG. 2D shows a cell 750 of a middle flow diversion zone 701, with pitch P1. The pitch P3 of the end zones 703, 705 can be the greatest and the pitch P1 of the middle flow diversion zone 701 can be the smallest with the pitch P2 of the intermediate density zones 702, 704 being in between so that P3>P2>P1. The lower material coverage of the zones outside the middle zone 701 allows for the flow diverter 700 to effectively treat the aneurysm and prevent blood from flow into the diseased site of the vasculature at the middle zone while preventing the flow diverter 700 from blocking branch vessels, for example, near the end zones. In an implementation, P1 or the axial spacing within the middle flow diversion zone 701 can be about 0.25 mm-0.40 mm, P2 or the axial spacing within the intermediate flow diversion zones 702, 704 can be about 0.35 mm-0.050 mm, and P3 or the axial spacing within the end flow diversion zones 703, 705 can be about 0.45 mm-0.75 mm. The bridge length within the middle zone 701, intermediate zones 702, 704, and distal/proximal end zones 703, 705 can vary. The bridge length within the intermediate zones 702, 704 can be at least 120% of the bridge length of the middle zone 701. The bridge length within the distal/proximal end zones 703, 705 can be at least 125% the bridge length of the middle zone 701, preferably about 150-300% (i.e., 1.5×-3×) the bridge length of the middle zone 701.

Strut width Wₛ, strut circumferential width Y, and strut axial length X can be the same in all three zones or can vary from one zone to another to vary the pattern density and physical characteristics between the zones. The distal and proximal zones 703, 705 can have one or two axial repetitions or rings 760, the intermediate zones 702, 704 can have two or three axial repetitions or rings 760, and the middle zone 701 can have 10-30 axial repetitions or rings 760.

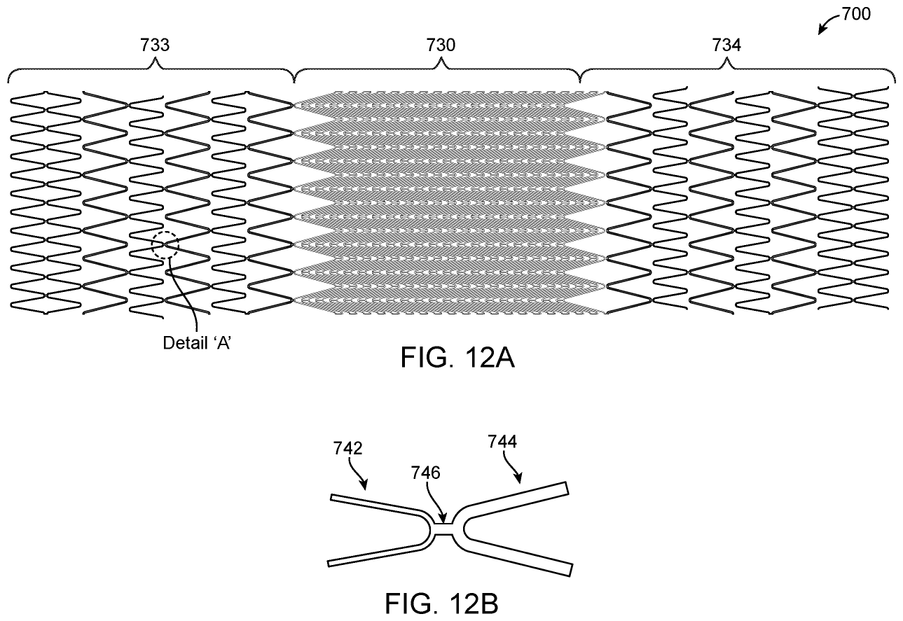

In an interrelated implementation, the flow diverter 700 may have a different cut pattern at either end to optimize the deployment characteristics resulting in an overall asymmetric design. FIG. 12A shows a flow diverter 700 having a middle flow diversion zone 730 having a cut pattern similar to middle flow diversion zone 701 of the flow diverter 700 in FIG. 1C. In contrast to the flow diverter 700 shown in FIG. 1C, the proximal end zone 733 and distal end zone 734 of the flow diverter 700 of FIG. 12A has a more traditional “stacked wave” pattern of conventional stents. The pattern of end zones 733 and 734 can be similar to intracranial stents, which are not designed for flow diversion but rather for supporting coiling procedures similar to, for example, the Stryker NEUROFORM ATLAS Stent. These stents are known to be easily and accurately deployed, in contrast to braided flow diverters. The “stacked wave” pattern has fewer connections between sections compared to the flow diverter shown in FIG. 1C and therefore opens more quickly during deployment. In other words, the flow diverter 700 of FIG. 12A achieves full expanded diameter when less of the expandable length is exposed and can therefore be “anchored” to the wall during the bulk of the deployment step. The first circumferential ring in the proximal end zone 733 can have peaks that connect with corresponding valleys of the second circumferential ring in the proximal end zone 733 forming fully closed cells at an outermost region in the proximal end zone 733. Similarly, the first circumferential ring in the distal end zone 734 can have valleys that connect with corresponding peaks of the second circumferential ring in the distal end zone 734 forming fully closed cells at an outermost region in the distal end zone 734. Thus, the first two outer rings of both the distal end zone 734 and the proximal end zone 733 can be fully closed diamond-shaped cells.

Still with respect to FIG. 12A, moving inward towards the middle zone 730, each of the proximal end zone 733 and the distal end zone 734 can have alternating larger and smaller rings. One inner circumferential ring in the proximal end zone 733 can be larger than the first two circumferential rings forming the diamond-shaped cells and also larger than the next circumferential ring positioned inward from the proximal-most end of the device. Similarly, one inner circumferential ring in the distal end zone 734 can be larger than the first two circumferential rings forming the diamond-shaped cells and also larger than the next circumferential ring positioned inward from the distal-most end of the device. This pattern can continue so that both the proximal end zone 733 and the distal end zone 734 have alternating larger and smaller circumferential rings moving inward toward the middle zone 730. The larger circumferential ring can have peaks and valleys that are larger in height as well as larger in circumferential span. Every other larger peak connects with every fourth smaller valley of the neighboring smaller circumferential ring. This results in every other peak of the larger circumferential ring remaining unconnected to the adjacent circumferential ring so as to form an open cell design. The circumferential ring on both the proximal and distal sides of the middle zone 730 can meet peak to valley with the neighboring peaks and valleys of the middle zone 730 circumferential ring forming closed diamond-shaped cells on either end.

The strut pattern can be varied even within end zones 733 and 734, to optimize the balance between device flexibility and radial strength. For example, as most easily seen in FIG. 12B (Detail A of FIG. 12A), some sections may have thinner struts 742 with a smaller “wave length” and adjacent sections may have wider struts 744 with a larger “wave length”. In an embodiment, the thinner struts have a width of 0.025″ (about 0.64 mm) and 12 waves around the circumference, whereas the thicker struts 744 have a width of 0.047″ (about 1.2 mm) and 8 waves around the circumference. Other strut dimensions and frequency may also satisfy the design requirements.

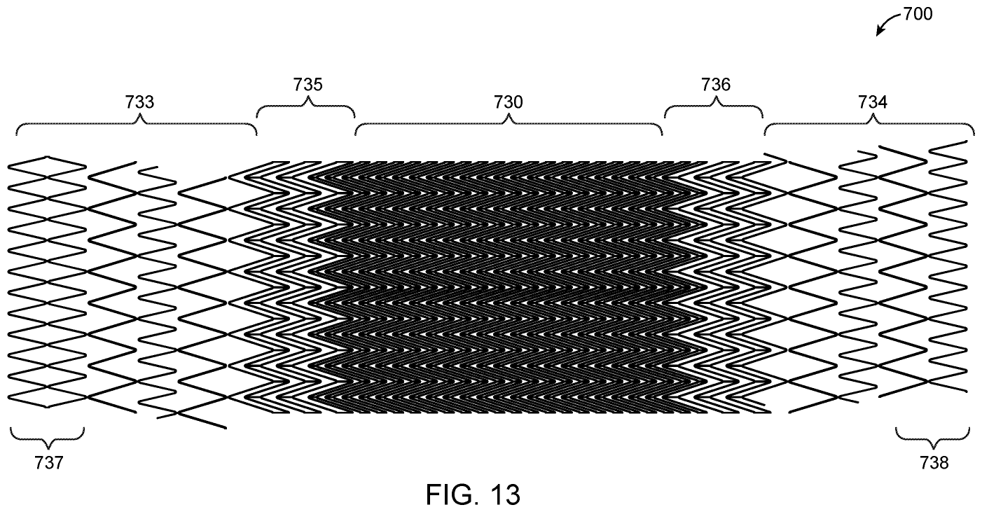

Further variations may be made along the length of the flow diverter 700. For example, FIG. 13 is a flat view of a flow diverter 700 having differences in strut pattern from one end of the flow diverter 700 to another end. The flow diverter 700 can include a first transition section 735 between the middle flow diversion zone 730 and the distal end zone 734. The flow diverter 700 can include a second transition section 735 between the middle diversion zone 730 and the proximal end zone 733. The transition sections 735 can have a pattern density in-between the dense middle flow diversion zone 730 and more open pattern density of end zones 733, 734. Transition sections 735 can have a 24-strut ring incorporated between the middle flow diversion zone 730 and the end zones 733, 734. The transition nested struts can provide increased spacing in the proximal and distal regions of the middle flow diversion zone 730.

The design of the distal end can differ from the design of the proximal end of the flow diverter. For example, as seen in FIG. 13, the distal end region 738 of flow diverter 700 formed by the outermost two circumferential rings has an “open-cell” design in which the end row of struts are only attached to the adjacent row of struts every 3 waves. In other words, the larger circumferential ring can have peaks and valleys that are larger in height as well as larger in circumferential span so that every other larger peak connects with every fourth smaller valley of the neighboring smaller circumferential ring resulting in every other peak of the larger circumferential ring remaining unconnected to the adjacent circumferential ring so as to form an open cell design. In contrast, the proximal end region 737 of flow diverter 700 formed by the outermost two circumferential rings has a “closed cell” design, in which every wave is attached to the adjacent strut peak-to-valley forming diamond-shaped cells. The open-cell nature of the distal end region 738 will make the radial expansion of the flow diverter 700 even more rapid during deployment, as the end row of struts is less connected to the adjacent row. The asymmetry between the distal and proximal ends can be achieved by having one end with a diamond shape and another end having a single 24-strut ring.

The width, length, number, and location of the axial connection struts can vary, as in axial strut 746 (shown in FIG. 12B) in the distal end zones 734 of flow diverter 700, or axial strut 752 (shown in FIG. 1F) in the middle section of flow diverter 700, to further optimize device flexibility with longitudinal stability. A major drawback of braided flow diverters is their tendency to shorten during expansion. A judicious addition of axial struts can maintain longitudinal stability of the device during deployment without making the device overly rigid.

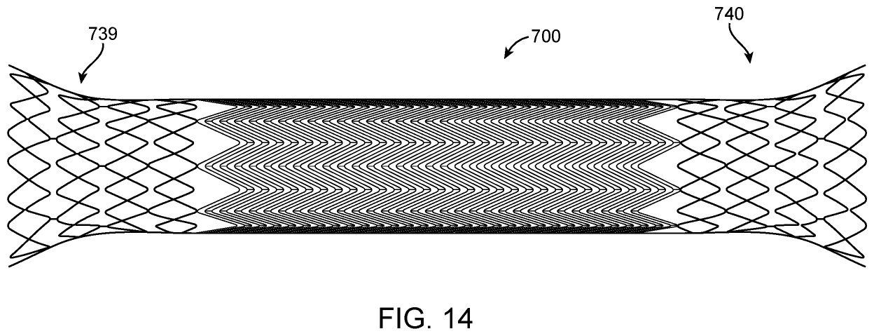

The ends of the flow diverter can be flared during manufacture of the device. For example, as seen in FIG. 14, flow diverter 700 has flared distal end 740 and flared proximal end 739. In this example, the distal end 740 has a more “open cell” construction such that gaps exist between some peaks. In another implementation, the distal end 740 and proximal end 739 have the same design whether closed cell, open cell, or hybrid design. The flare angle may vary from 15 degrees to 40 degrees, or about 20 to 30 degrees relative to a longitudinal axis of the flow diverter 700 from the proximal end to the distal end. The purpose of the flared shape is to ensure good apposition of the flow diverter ends to the vessel wall even if the device is deployed in a curve. The springy nature of nickel titanium (NiTi) cut tube devices causes the device with flared ends when positioned within a curve to press against the outside of the curve. A device with no flared ends may lift off the vessel wall on the inside of the curve. Either end of the device, depending on the position of the device in the curve, can lift off the wall. If the end is lifted off, it may cause problems with subsequent device advancement or retraction through the flow diverter, as well as increase the risk of device thrombosis.

The flare at the proximal end of the flow diverter can be greater than the flare at the distal end of the flow diverter. For example, the angle of the flare at the proximal end can be about 40 degrees and the angle of the flare at the distal end can be about 20 degrees such that the diameter of the opening into the stent lumen on the proximal end can be about 10 mm and the diameter of the opening into the stent on the distal end can be about 7 mm. The outer diameter within a central region of the stent can be about 4.25 mm, in comparison. The length of the flare on the distal and proximal ends can each be about 3-4 mm long and the non-flared uniform OD region can be about 20-23 mm long.

The length of middle flow diversion zone 730 can be about 8 mm-12 mm. The length of each of the end zones 733, 734 can be about 5 mm-10 mm for a total length of about 18 mm-35 mm, preferably about 20 mm-30 mm. The number of nesting struts around the circumference of the stent can be greater than 12 such as about 16 so as to align with 16-strut geometry of the anchor region. The gap between struts in the middle flow diversion zone can be about 33-34 microns.

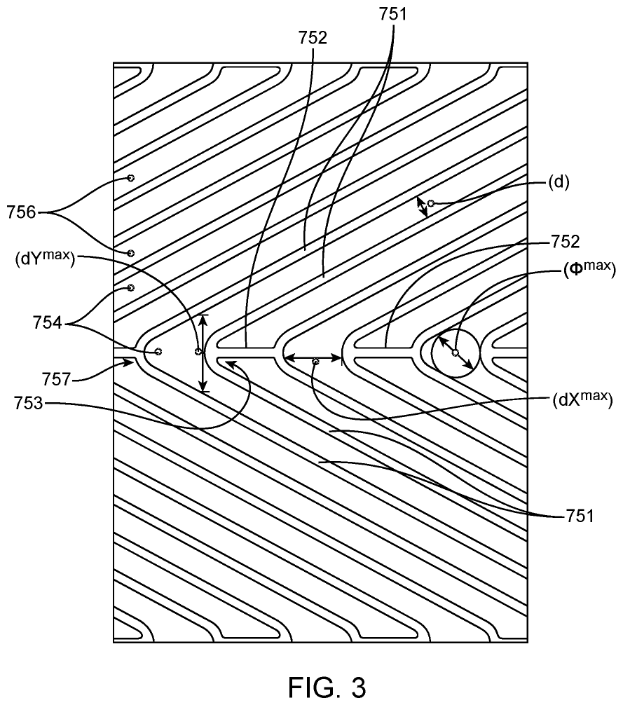

FIG. 3 shows details of the repeating laser cut pattern of the flow diverter 700, illustrating how porosity of the device can be defined. In this view, the V-shaped cells 750 are nesting within one another so that the central bends are aligned and the peak 757 of one cell 750 is spaced from the valley 753 of the adjacent cell 750 by the distance of the connecting bridge 752. The struts 751 of the cell 750 define a V-shaped opening 754 therebetween. The spacing between the cells 750 provided by the bridge 752 forms additional V-shaped openings 756 that are arranged in reverse of the cells 750. The openings 754 of the cells 750 and the openings 756 between the cells 750 (when observing the flow diverter in an inverse orientation) represent open space (AO), and the struts 751 and bridges 752 represent the metal structure of the flow diverter (AM). The sum of the open space and the metal structure is equal to the area of the bounding rectangle, “A”. Porosity (p) can be defined as the ratio of the open space to the total area, p=AO/A. Coverage (c) can be defined as the ratio of metal area of total area, c=AM/A. In the present configuration, porosity is approximately equal to 70% and material coverage is approximately equal to 30%. The typical gap between adjacent struts 751 of a cell 750, shown in FIG. 3 as d, can vary depending on whether the cells 750 are located within the middle zone 701, an intermediate zone 702, 704, or an end zone 703, 705 of the flow diverter 700. In some implementations, the gap d between strut pairs 751 of a single cell 750 in the middle zone 701 can be approximately 0.08 mm. The coverage and porosity described is calculated for the middle zone 701 of a flow diverter with the following dimensions at the expanded configuration illustrated in FIG. 3. The gap between the central bends of strut pairs 751 of the single cell 750 is shown in FIG. 3 by arrow labeled dX^{max}. This is the maximum gap between strut pairs in the direction of flow and can be approximately 0.22 mm in the middle zone 701 of the flow diverter 700. The maximum gap between strut pairs 751 of a single cell 750 perpendicular to the direction of flow is shown in FIG. 3 by arrow labeled dY^{max} and can be approximately 0.29 mm in the middle zone 701. The area of the V-shaped opening 754 at the central bends of the strut pairs (e.g., between the valley 753 and peak 757) of a cell 750 can be represented by a maximum diameter Φ^{max} of a circle that fits within the opening 754. This maximum diameter Φ^{max} can be approximately 500 microns or less, or between about 100 microns and 500 microns, preferably about 250 microns for a cell 750 within the middle zone 701. The size range of the gap d, the dX^{max}, the dY^{max}, and the Φ^{max} of a cell 750 can vary depending on whether the cell 750 is within the middle zone 701, the intermediate zone 702, 704, or the end zones 703, 705. The dX^{max}, dY^{max}, and Φ^{max} of a cell 750 can be about 500 microns or less, about 100 microns and 500 microns, such as about 250 microns.

The flow diverters described herein can incorporate radiopaque marker receptacles 770 (see FIG. 1B) to one or more of the end features and/or features located. For example, a first set of one or more receptacles 770a can be positioned at the proximal and distal ends of the flow diverter and a second set of one or more receptacles 770b can be positioned to identify the length Lc such as at either end of middle flow diversion zone 701. The receptacles 770a can be positioned at the distal-most and proximal-most ends whereas receptacles 770b can be positioned on the valleys of the middle flow diversion zone 701 to identify that densest coverage. Radiopaque material can be pressed into the receptacles 770 to make the ends and zones of the flow diverter 700 visible under fluoroscopy. For example, radiopaque markers can be located in receptacles 770b on either end of the middle zone 701 so that the user is able to confirm the location of the middle zone 701 with approximately 30% material coverage is appropriately located across the aneurysm neck, and/or perform procedural steps to ensure that this is true. The arrangement of the receptacles 770 can vary and a few are shown in FIG. 1B as an illustration and is not intended to be limiting as the receptacles 770 can be positioned in any of a variety of locations depending on which portions of the flow diverter are desired to be visualized. At least one of the proximal end zone, the middle zone, and the distal end zone can include at least one radiopaque marker.

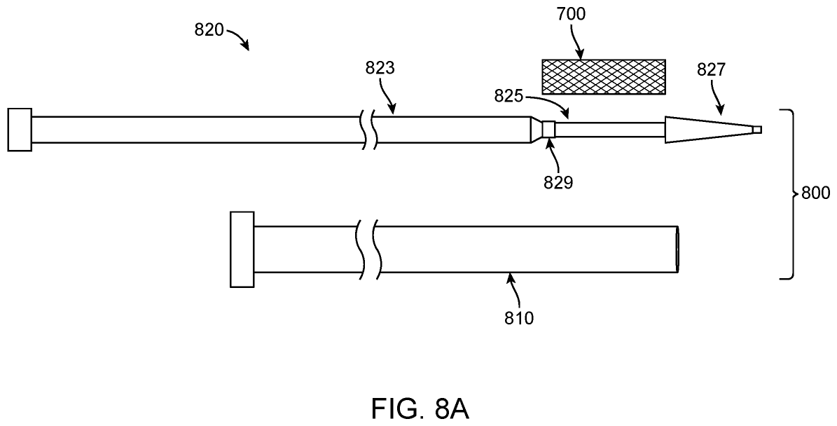

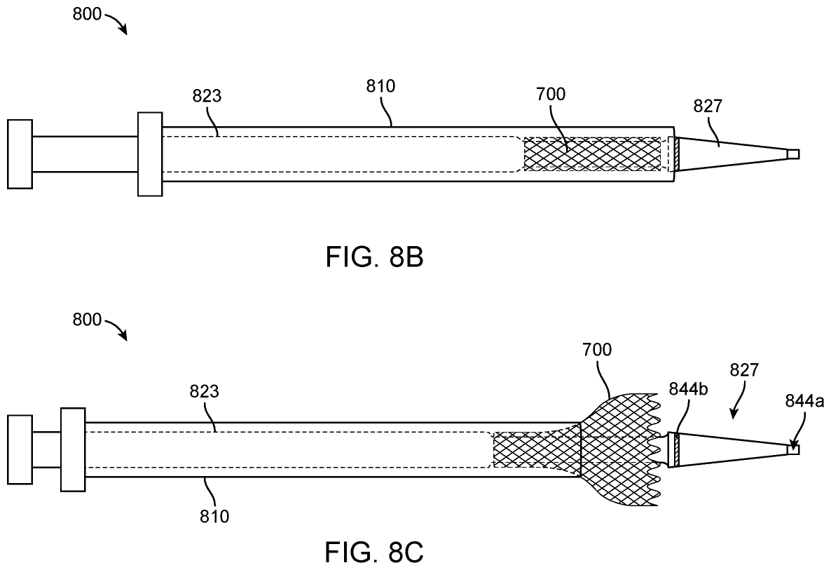

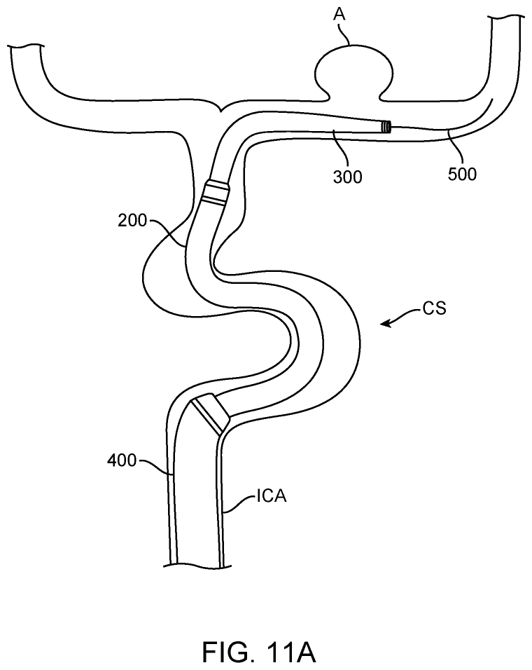

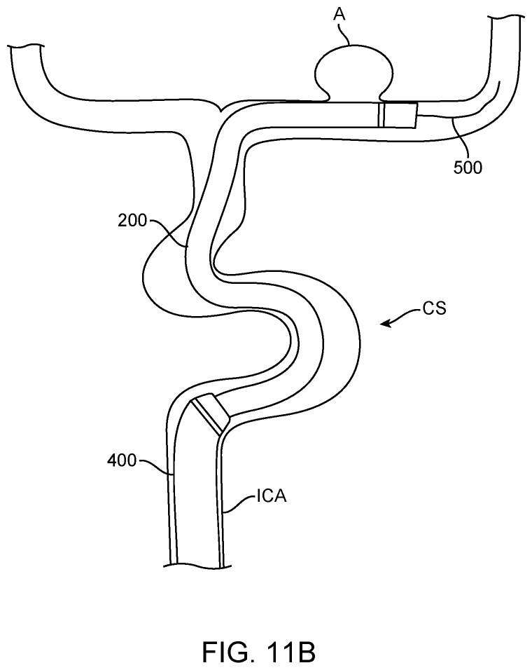

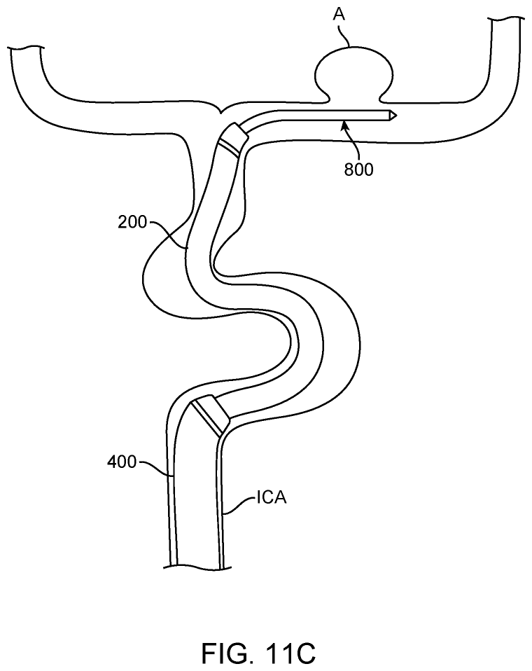

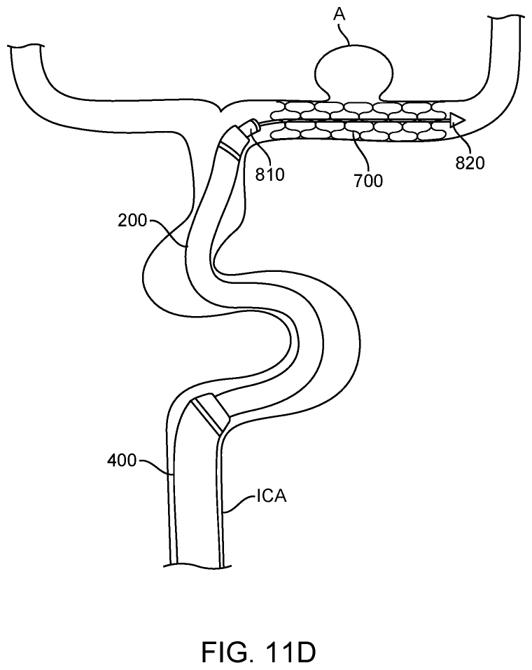

The flow diverter 700 can be designed to be at least partially re-sheathable for some distance during the deployment process. As will be described in more detail below with reference to FIGS. 8A-8C and also FIGS. 11C-11D, the flow diverter 700 can be deployed using a flow diverter delivery system 800 having an inner core member 820 and an outer restraining sleeve 810. The flow diverter 700 is mounted on the inner core member 820 and the outer restraining sleeve 810 can be pulled back a specified amount to partially deploy flow diverter 700 (see FIG. 8C). The flow diverter 700 design allows a user to withdraw the restraining sleeve 810 a distance proximal to partially deploy a portion of the flow diverter and then re-advance the sleeve distally, if desired, to relocate a distal end 707 of the flow diverter 700.

The cut pattern of the flow diverter 700 determines if the flow diverter is re-sheathable. If the pattern contains features that pop open beyond the inner diameter of the outer restraining sleeve 810, the flow diverter 700 generally cannot be re-sheathed after partial deployment. However, the flow diverters described herein can incorporate features (see rail 775 of FIG. 1E) that ensure the flow diverter 700 stays within a particular outer diameter that can be received within the restraining sleeve even after partial deployment.

The flow diverter of FIGS. 1A-1C have bridges 752 connecting the valley 753 of a first cell 750 to a peak 757 of a cell 750 in the same row 765 of the adjacent ring 760. These peak-to-valley connections, however, can skip a ring 760 so that these peak-to-valley bridges alternate. The bridges 752 connecting the struts 751 near the edges of the cells 750 also alternate from ring to ring. In the implementation of the flow diverter in FIGS. 1D-1F some bridges 752 connecting the struts 751 near the edge of the cells do not alternate from ring to ring. Instead, these bridges 752 form a continuous rail 775 extending an axial length of the flow diverter 700. The rail 775 can form a line connecting radially adjacent hinges (or central bends) passing through at least 3 cells, at least 4 cells, at least 5 cells, at least 6 cells, at least 7 cells, or at least 8 cells. The line can connect 3-8 cells or about 4-6 cells. The cells being connected can be found within the middle zone, the intermediate zone, or an end zone. The line connecting radially adjacent hinges can be in the proximal and distal zones and pass through fewer cells than in a line connecting radially adjacent hinges in the middle zone. The rails 775 can be found within one or both zones 702, 703 near the distal end 707 of the flow diverter 700 and not within corresponding zones 704, 705 near the proximal end 709 of the flow diverter 700 such that the flow diverter 700 has an asymmetry between its distal end 707 and proximal end 709. The presence of the rails 775 within zones 702 and/or 703 near the distal end 707 of the flow diverter 700 is configured to allow for re-sheathing even after a restraining sleeve 810 is withdrawn to partially deploy zones 702, 703 and expose at least a portion of the middle zone 701. In yet another implementation, these additional axial rails 775 can be incorporated within one or more other zones along a length of the device (e.g., zones 703, 702, 701, 704 and 705) to make the flow diverter 700 resheathable over almost a complete deployment length of the flow diverter 700. Alternately, the axial rails 775 can extend over one end region 703 of the flow diverter rather than two or more regions of the flow diverter 700 if more flexibility is desired over the ability to re-sheath. The axial rails 775 are shown extending between peak-to-valley portions of the cells 750. Other axial features can be added at other location(s) of the cells 750 such as between struts. The axial peak-to-valley rails 775 or other axial features between struts added between ring segments 760 can also limit intrusion of features into a lumen or aneurysm space when the flow diverter 700 is positioned in a curve (a phenomenon termed “fish-scaling” when referring to open-cell stent designs).

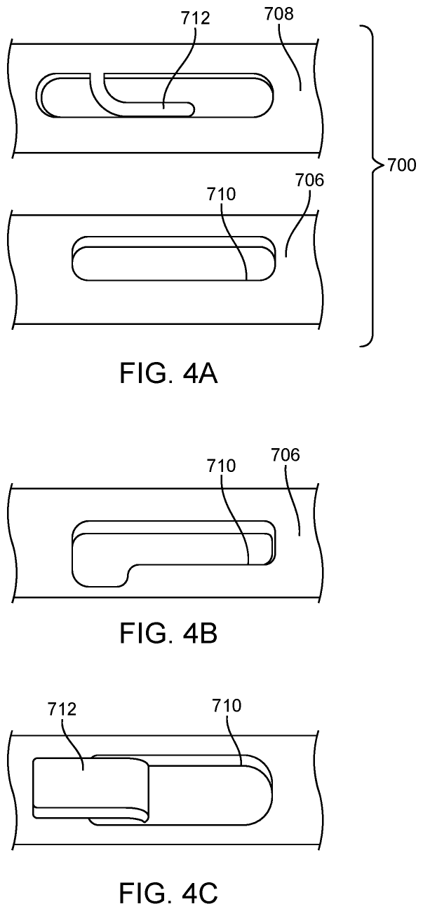

The implementations shown in FIGS. 1A-1F have a cut pattern that provides the desired material coverage upon expansion in the vessel, a coverage that can (but need not) change along an axial length of the flow diverter. FIGS. 4A-4C illustrate a flow diverter 700 constructed from two laser-cut non-braided tubular members or tubes 706, 708 that together provide the desired material coverage when the flow diverter is expanded in the vessel and depending on the overlap can provide progressive material coverage along a length of the flow diverter 700. Each of the two tubes 706, 708 may each have a density of 15% when expanded, but when assembled together have a total material density of 30%. The two tubes 706 and 708 may be assembled to be overlapping but staggered, to create an overlap region having a first density (e.g., 30% material coverage and 70% porosity) and each end of the staggered tubes 706, 708 having a second lower density (e.g., 15% material coverage and 85% porosity). The two tubes 706, 708 may be locked together with locking features built into the laser cut pattern. For example, as shown in FIG. 4A, one tube 706 may have one or more holes or elongate slots 710 laser cut into the tube 706 on either or both ends, and the second tube 708 may have one or more corresponding tabs 712 formed to protrude into the slot 710 and then lie flat. The two tubes 706, 708 are assembled such that the tabs 712 are inserted into the slots 710 and then the tubes 706, 708 are slid with respect to each other to lock the two tubes 706, 708 together. In a variation, as seen in FIG. 4B, the slot 710 may have an ‘L’ shape such that the two tubes 706, 708 can be rotated with respect to each other to lock the two tubes 706, 708 together. Alternately the tab 712 can be pushed through the slot 710 and bent to lock into place, as shown in FIG. 4C. The tabs 712 can be on the inner tube 708 and the slots 710 on the outer tube 706, or vice versa.

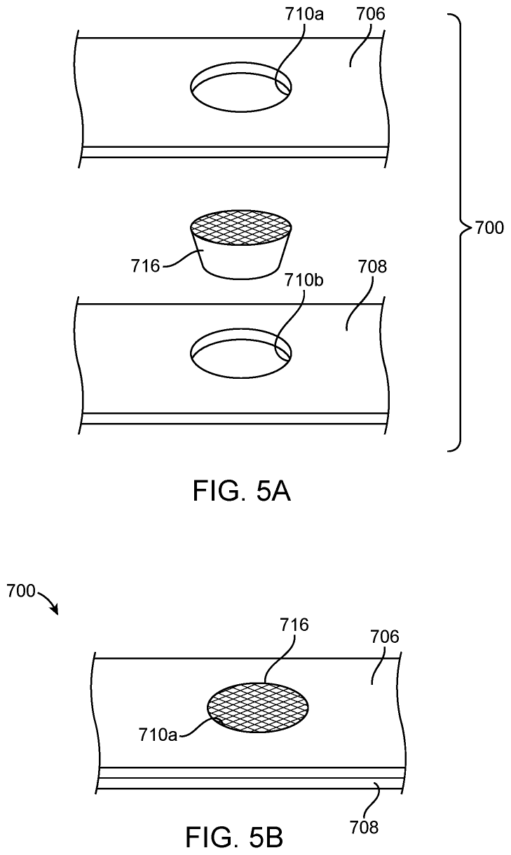

FIGS. 5A-5B illustrate another locking mechanism for a flow diverter 700 constructed from two laser-cut tubes 706, 708. Both tubes 706, 708 can be laser cut to include holes or elongate slots 710 on either or both ends in corresponding positions. The two tubes 706, 708 are assembled one inside the other so that their respective holes 710a, 710b are aligned. A disk 716 made from a malleable material can be pressed into the holes 710a, 710b to lock the tubes 706, 708 together. The disk 716 may be slightly tapered (i.e., from an upper side toward the lower side as shown in FIG. 5A) and sized such that when the disk 716 is pressed into place, the disk 716 deforms to fill the holes 710a, 710b and is held securely in place. The disk 716 can be a radiopaque malleable material, such as gold, gold alloy, or tungsten to serve both as a radiopaque marker on the implant as well as a locking mechanism.

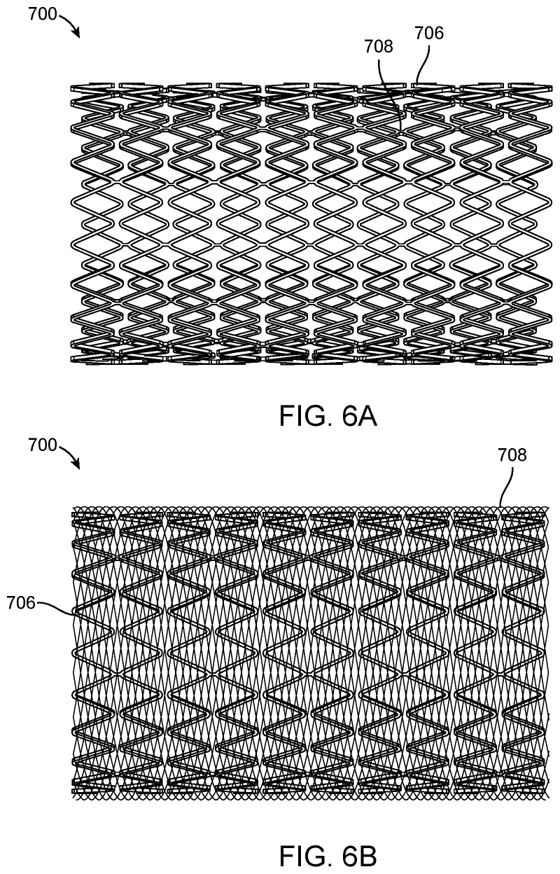

FIG. 6A illustrates another implementation of a flow diverter 700 constructed from two laser-cut tubes 706, 708. One tube 706 is designed to provide structural integrity to the flow diverter 700, for example, to provide full wall apposition and anchoring, such as by a wall thickness and/or strut width. The other laser cut tube 708 is designed to provide the 30% material coverage and has a very fine strut pattern and thin wall thickness. The finer cut tube 708 may also be a very fine-wire braided tube, or a porous material such as an expanded PTFE tube, as seen in FIG. 6B. The finer strut pattern tube 708 may also be shorter than the larger strut structural tube 706. Alternately, as shown in FIG. 6B, the two tubes 706, 708 may be the same length and the tubes 706, 708 substantially overlap each other.

These multi-layer flow diverter implants 700 utilize the structural stent layer of tube 706 to provide precise placement and anchoring, and the finer stent layer of tube 708 to provide the higher material coverage that diverts the blood from flowing into the excluded aneurysm. The larger-diameter access systems described herein enable delivery of these multi-layer devices, which would not be possible in the current microcatheter delivery methods having smaller inner diameters (e.g., 0.027″), which as described above, are incapable of accommodating a laser-cut flow diverter alone and certainly not a flower diverter plus a restraining sleeve.

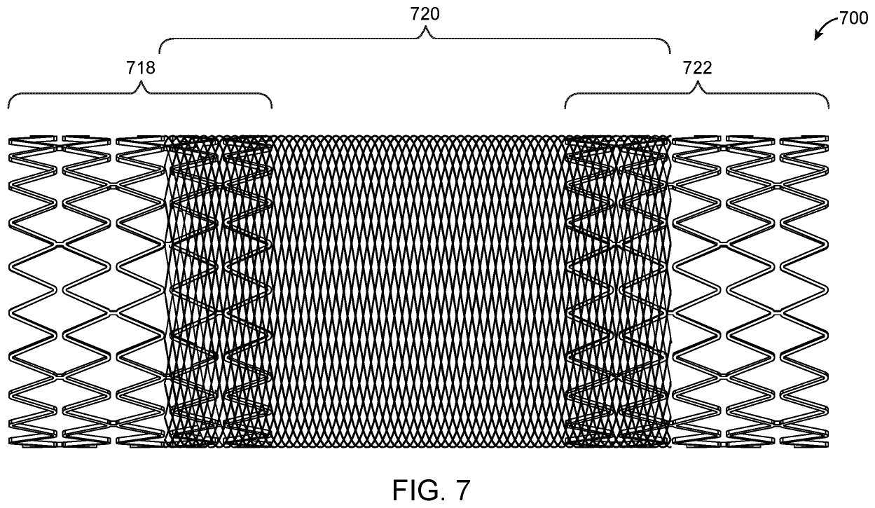

The flow diverter 700 can also be made of varying materials and structures along its length. For example, as shown in FIG. 7, the flow diverter 700 is formed of two laser cut bands 718, 722 on both ends of the device. A finer structure, such as a braided wire tube 720, can be positioned between the two laser-cut bands 718, 722 and connected to the laser-cut bands to form a multi-segment implant. The braided wire tube 720 can be interlaced with the laser-cut bands 718, 722 to couple to the bands. This compound or hybrid design provides two end anchors to the flow diverter 700 with the higher material coverage across the aneurysm.

The flow diverter implants described herein can be self-expanding tubes or tube components cut to achieve a desired pattern. Any of the cut-tube components in the flow diverters described herein may be self-expanding materials or manufactured from one or more Nitinol laser-cut tubes. The tubes can be Nitinol or another spring material capable of the desired mechanical properties of the self-expanding device.

The tubes or cut tube components can be cut with lasers, mechanically machined, photo-etched by photolithography, or other chemical etching, and the like to achieve the desired cut pattern. As an alternative to cutting the design from cylindrical tubing, the design may be cut or assembled in a planar configuration as a flat pattern and compressed or otherwise wrapped or rolled up into a spiral or cylindrical configuration for delivery, and expanded in situ into a partially cylindrical configuration, a cylindrical configuration, or a partially or fully overlapping roll configuration. In the latter implementation, features might be included to latch or ratchet the flow diverter in the expanded shape. The cut tube could also be manufactured by vapor deposition of material in a tube or flat pattern, the latter to be rolled up.

The cut tubes can undergo finishing processes, such as electropolishing and heat-setting to achieve desired mechanical and dimensional properties. In some implementations, the flow diverter can be heat-shaped to have one or both ends 707, 709 flared to aid in anchoring of the flow diverter to the vessel wall during deployment.

Other materials and manufacturing methods can also be utilized to manufacture flow diverters, as described herein. Alternately, any of the above flow diverter implants may be a balloon-mounted laser cut stents, manufactured from one or more laser-cut stainless steel, cobalt-chromium alloy, or other materials known to be used for balloon-expandable stents. The flow diverters can be fabricated from tubing material including radiopaque materials in addition to the typical constituents of superelastic nickel titanium. For example, the radiopaque material can include platinum, tantalum, tungsten, or gold. The radiopaque material can be homogenously incorporated into the material in an advantageous proportion, or the material can be constructed as a laminate including one or more layers of radiopaque material in addition to one or more layers of nickel titanium, or coated onto the surface of the nickel titanium.

The systems described here is used with a larger-bore access system, and therefore, if desired, the flow diverter may be a braided-wire-style flow diverter in which the braid parameters of the braided-wire-style flow diverter are modified to improve performance. For example, the wire size and/or number of wires can be increased without the design restriction of being deliverable through a 0.021″ or 0.027″ ID microcatheter delivery system as is required with current flow diverters. An example of a current braided flow diverter is the Pipeline Embolization System with 48 wires×30 microns (0.0013″). An increase in wire size would make it incompatible with the 0.027″ ID microcatheter. Flow diverters with higher numbers of braid wires have smaller wire sizes, for example the Surpass Evolve has a 64-wire braid with 0.0011″ wire. Again, an increase in wire size would make it incompatible with the 0.027″ ID microcatheter. In an implementation, the flow diverter 700 is a braided-wire flow diverter constructed from 48 or up to 96 wires or more, with strands of between 35 and 55 micron diameter. These heavier-gauge and/or larger number of wire strand braided flow diverters have a heavier radial force and spring open with more speed than the currently available flow diverters, making them easier to deploy and reducing current issues with braided flow diverters, such as flattening and ribboning during deployment

The flow diverters described herein may have a specialized antithrombotic surface modifications or coatings, for example, heparin coatings, hydrophilic polymer coatings, such as phosphorylcholine and phenox hydrophilic polymers, albumin, fibrin, and the like.

Flow Diverter Delivery Systems

Flow diverters are conventionally mounted on an inner delivery core wire and delivered through a microcatheter having an inner diameter of 0.027″ (0.7 mm). In order to be delivered through such a small-sized delivery system while still providing the desired wall coverage (approximately 30%) when expanded in vessel up to 5.0 mm diameter, flow diverters conventionally have braided wire construction.

The delivery of conventional braided flow diverters typically occurs over several procedural steps. First, a microcatheter is inserted into the vasculature and advanced over a guidewire to a position across the target aneurysm site. The microcatheter tip is often placed far distal to the ultimate target implant site because of the imprecise nature of delivering braid-style flow diverters. Once the microcatheter is in position relative to the target aneurysm, the guidewire is removed. The braided flow diverter is then inserted to the proximal end of the microcatheter using an introducer tube. The flow diverter is pre-mounted on a delivery core wire with features to keep the flow diverter both restrained in the collapsed configuration and secured longitudinally onto the delivery core wire. For example, the core wire can have PTFE sleeves that cover and constrain the braided flow diverter at either end. The core wire often has a distal flexible tip that extends up to 15 mm beyond the distal end of the flow diverter. This means that the distal tip needs to be positioned at least 15 mm beyond the treatment site, and possibly more if the microcatheter is positioned distally, for the flow diverter to be implanted in the correct location, another source of potential complication. The core wire is used to push the flow diverter to the end of the microcatheter. The microcatheter is then retracted to expose the braid, which, by its material properties and construction, begins to spring open. The distal end does not reach its full opening diameter until several millimeters of the braid are exposed due to the nature of the braided construction. The user must often push on the microcatheter while pulling on the core to “push” the braid to its maximum opening in order to get full apposition of the flow diverter against the vessel wall, which is highly desirable to achieve the intended clinical effect. This push and pull technique is yet another potential cause of clinical complication of conventional braided flow diverters as well as adding time to the procedure and imprecision in the implantation location. Braids by their nature shorten considerably upon expansion, making accurate implantation yet more difficult. Often, the flow diverter is delivered distal to the desired site and then partially deployed and “dragged back” into place across the target site. Both the distal positioning of the microcatheter and the “drag back” step are risks for vessel damage and vessel perforation, both leading to severe clinical sequelae.

In many flow diverter delivery systems, the delivery core wire has features that constrain the braid wire ends. The microcatheter following expansion of the flow diverter is fully proximal to the implant and must be re-advanced through the braid to cover the delivery core wire features so that the delivery core wire does not get snagged by the just-deployed flow diverter. Each of these steps potentially disrupt the flow diverter, add to procedural time, and are potential causes of clinical complications due to the extra catheter maneuvering.

The flow diverters described herein can be delivered by flow diverter delivery systems that are larger in diameter and configured to be used with larger-bore access systems compared to conventional braided-style flow diverters. The delivery systems described herein can be used with any of the above flow diverters described previously including laser cut, braided, or woven flow diverters, or combinations thereof.

FIGS. 8A-8C illustrate a flow diverter delivery system 800 having an outer restraining sleeve 810 and an inner core member 820 having an elongate shaft 823. The inner core member 820 can have an inner lumen (not shown) sized to accommodate a guidewire. The lumen can be a single, central lumen that allows the flow diverter 700 and flow diverter delivery system 800 to be delivered over a guidewire. The shaft 823 of the inner core member 820 has a reduced diameter recessed section 825 near a distal end region that is sized to accommodate a flow diverter 700. As shown in FIG. 8B, the flow diverter 700 is positioned in the recess 825 of the inner core member 820 and is retained in this position by the outer restraining sleeve 810. The flow diverter 700 is held by the inner core member 820 within the recessed section 825 and deployed by expansion upon withdrawing the restraining sleeve 810 proximally. The inner core member 820 can include a grip feature 829 located at a proximal end of the recessed section 825 that is configured to prevent the flow diverter 700 from being dragged back over shaft 823 of the inner core member 820 as the restraining sleeve 810 is withdrawn during flow diverter deployment. The grip feature 829 can be a high friction component, such as a length of thin-walled silicone or other elastomeric tube.

The materials of the shaft 823 of the inner core member 820 are selected to maintain axial integrity during deployment of the flow diverter 700. For example, the shaft 823 and recessed section 825 can be constructed from Pebax, such as Pebax 72D. The shaft 823 and/or recessed section 825 can be braid-, coil-, or otherwise reinforced to provide axial stiffness.

The length of the outer restraining sleeve 810 is shorter than the inner core member 820 by an amount that allows the flow diverter 700 to be fully deployed when the restraining sleeve 810 is pulled back with respect to the inner core member 820 (see FIG. 8C). The restraining sleeve 810 is configured so that it is able to be pulled back easily without dragging the flow diverter 700 with it. For example, the restraining sleeve 810 can be constructed with multiple layers including a low friction inner liner, such as PTFE or FEP. The restraining sleeve 810 can be braid- or coil-reinforced so as not to stretch during withdrawal. The restraining sleeve 810 can also have an outer hydrophilic coating on the distal portion to improve delivery through a large-bore catheter, which will be described in more detail below.

Again with respect to FIG. 8A, the inner core member 820 can include a distal tip region 827 located distal to the recessed region 825. The distal tip region 827 of the inner core member 820 is tapered and has a flexibility, shape, taper length and taper angle configured for atraumatic delivery of the delivery system 800 to a vessel in the brain with or without a guidewire. The construction, materials, and configuration can be similar to the tapered tip 346 of catheter advancement element 300 described below with respect to access system 100 and described in U.S. Pat. No. 11,065,019 which is incorporated herein by reference in its entirety. For example, the distal tip region 827 can have two radiopaque markers 844a, 844b configured to delineate the tapered section. A first radiopaque marker 844a can identify the distal-most end of the inner core member 820 and a second radiopaque marker 844b can identify a maximum outer diameter region of the taper for optimum delivery purposes relative to the outer restraining sleeve 810. The outer diameter of the inner core member 820 just proximal to the taper is sized to be a smooth fit against the inner diameter of the restraining sleeve 810 so as to present a smooth leading edge to the flow diverter delivery system 800 being advanced in the vasculature with or without a guidewire.

The dimensions of the flow diverter 700 and the flow diverter delivery system 800 are sized to be deliverable through a larger-bore access systems. As discussed above, the flow diverter 700 can be a cut-tube design having cells 750 arranged in rings 760 that are connected peak-to-valley. Upon expansion, the flow diverter 700 has a dense material coverage (e.g., 30% coverage or 70% porosity) due to the tightly nested arrangement of the cells 750. The flow diverter 700 can take advantage of the constraint of a larger-bore access system to achieve this dense material coverage. For example, for a distal access system 100 having an access catheter 200 with inner diameter (ID) of 0.088″, the outer restraining sleeve 810 can have an outer diameter (OD) of about 0.082″ leaving an annular clearance of 0.003″ (ID/OD difference of 0.006″) for optimal advancement of the flow diverter delivery system 800 through the access catheter 200. In this example, the ID of the outer restraining sleeve 810 is about 0.070″. The collapsed flow diverter 700 can have an OD of about 0.064″ in order to slide easily through this outer restraining sleeve 810. The inner core member 820 can have an OD of about 0.064″, with the smaller ID recessed section 825 depending on the wall thickness of the flow diverter 700. If the wall thickness of the cut-tube flow diverter 700 is about 0.005″, the recessed section 825 has an OD of about 0.054″.

Larger access systems allow for alternate delivery methodologies. For example, rather than first placing a microcatheter across the aneurysm, removing the guidewire, and then pushing the flow diverter into place as with conventional flow diverter delivery systems, the flow diverters 700 described herein can be pre-mounted onto the delivery system 800 with the restraining sleeve 810, and delivered to the site through a larger delivery system (e.g., 0.087″-0.126″ ID). The guidewire, flow diverter 700, and inner core member 820 can all be pre-mounted in one system rather than exchanging the guidewire for the flow diverter and inner core member as in conventional systems.

In some implementations, the access catheter 200 acts as the restraining sleeve for the flow diverter delivery system 800 in place of a separate restraining sleeve 810. The flow diverter 700 can be mounted on the inner core member 820 and introduced into the access system 100 via a separate introducer component and pushed via advancement of the inner core member 820 to the target aneurysm treatment site in the same manner as current flow diverters may be introduced into microcatheters previously positioned across the target site. In this example, the access catheter 200 can be previously positioned across the target site. Once the flow diverter 700 is positioned at its target site, the inner core member 820 can be held in place while the access catheter 200 is pulled back to deploy flow diverter 700. In this example, there is one “layer” of catheters that is eliminated (i.e., the restraining sleeve 810). This allowed for a larger inner diameter for a same size flow diverter. The flow diverter having an outer diameter of 0.064″ can be delivered using an access catheter having an inner diameter of 0.070″ and an outer diameter of 0.082″ (vs. ID 0.088″ and OD 0.100″ of the previous example).

### Access Systems

The flow diverters and flow diverter delivery systems described above can be delivered using an access system and/or access catheter with an appropriately large-bore inner diameter and the ability to reach the target aneurysm treatment site. Current access devices, i.e., guide catheters and/or guide sheaths, are used to access neurovascular anatomy with limitations.

Guide catheters or guide sheaths are used to guide interventional devices to the target anatomy from an arterial access site, typically the femoral artery. The length of the guide is determined by the distance between the access site and the desired location of the guide distal tip. Interventional devices, such as guidewires, microcatheters, and intermediate catheters used for sub-selective guides, are inserted through the guide and advanced to the target site. Often, devices are used in a co-axial fashion, namely, a guidewire inside a microcatheter inside an intermediate catheter, and advanced as an assembly to the target site in a step-wise fashion with the inner, most atraumatic elements, advancing distally first and providing support for advancement of the outer elements. The length of each element of the coaxial assemblage takes into account the length of the guide, the length of proximal connectors on the catheters, and the length needed to extend from the distal end.

Typical tri-axial systems, such as for delivery of flow diverters, stents, stent retrievers and other interventional devices, require overlapped series of catheters, each with their own rotating hemostatic valves (RHV) on the proximal end. For example, a guidewire can be inserted through a Penumbra VELOCITY microcatheter having a first proximal RHV, which can be inserted through a Penumbra ACE68 having a second proximal RHV, which can be inserted through a Penumbra NEURONMAX 088 access catheter having a third proximal RHV positioned in the high carotid via a femoral introducer. Maintaining the coaxial relationships between these catheters can be technically challenging. The three RHVs must be constantly adjusted with two hands or, more commonly, four hands (i.e., two operators). Further, the working area of typical tri-axial systems for intracranial and cerebral device delivery can require working area of 3-5 feet at the base of the operating table. Time is required to access the treatment site using tri-axial systems.

There is also difficulty in getting larger-bore access catheters and sheaths in a rapid and atraumatic fashion to intracranial and cerebral vessels. Both the lengths and diameters of current systems put limitations on the delivery system of endovascular scaffolding devices, such as stents, or flow diverters, which in turn limits the safety, speed, and precision of delivering such devices. There is a need for a system of devices and methods that allow for rapid access of distal intracranial and cerebral vessels with larger lumen sizes and/or shorter lengths.

The access systems and methods described herein enable safe and rapid positioning of large interventional devices, such as flow diverter delivery systems, to an aneurysm site in an intracranial or cerebral artery. Further, the extreme flexibility and deliverability of the distal access catheter systems described herein allow the catheters to take the shape of the tortuous anatomy rather than exert straightening forces creating new anatomy. The distal access catheter systems described herein can pass through tortuous loops while maintaining the natural curves of the anatomy therein decreasing the risk of vessel straightening. The distal access catheter systems described herein can thereby create a safe conduit through the neurovasculature maintaining the natural tortuosity of the anatomy for other catheters to traverse (e.g. interventional device delivery catheters). The catheters traversing the conduit need not have the same degree of flexibility and deliverability such that if they were delivered directly to the same anatomy rather than through the conduit, would lead to straightening, kinking, or folding of the anterior circulation.

Provided herein are access systems including a catheter advancement element having a tapered distal end region with a flexibility, shape, and taper length configured to be atraumatically delivered to a vessel in the brain. This is not achieved with conventional catheter systems as they may have improper flexibility, are formed of improper materials, or have improper shape and/or taper length resulting in conventional catheter systems getting misdirected or hung up or, if more force is applied, perforating the vessel. Unlike these conventional catheter systems, the catheter systems described herein includes a catheter advancement element capable of safely navigating neurovascular anatomy and find the lumen so that a corresponding large bore catheter (i.e., flow diverter delivery system) can be delivered to distal sites. The catheter systems described herein help locate occlusions in the vessels in the novel manner of the methods provided herein. These and other features will be described in detail herein.

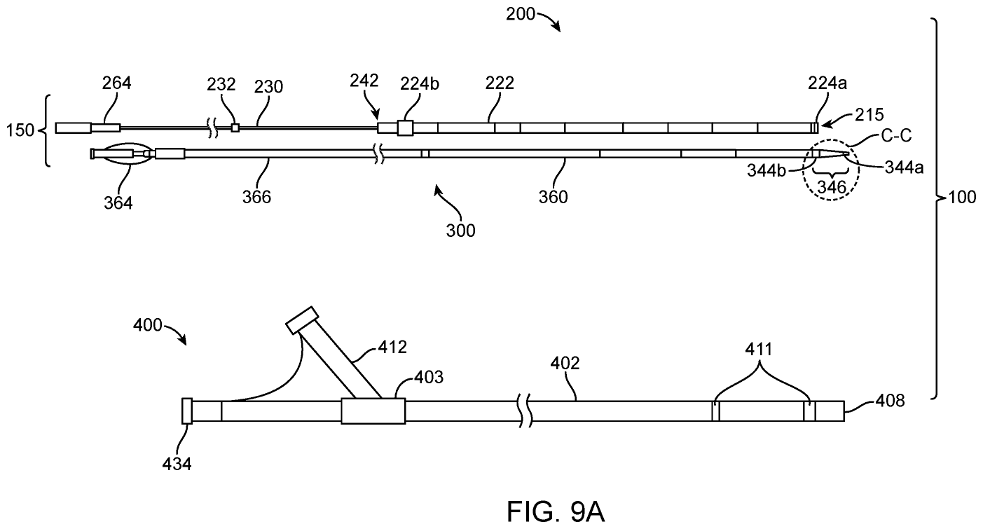

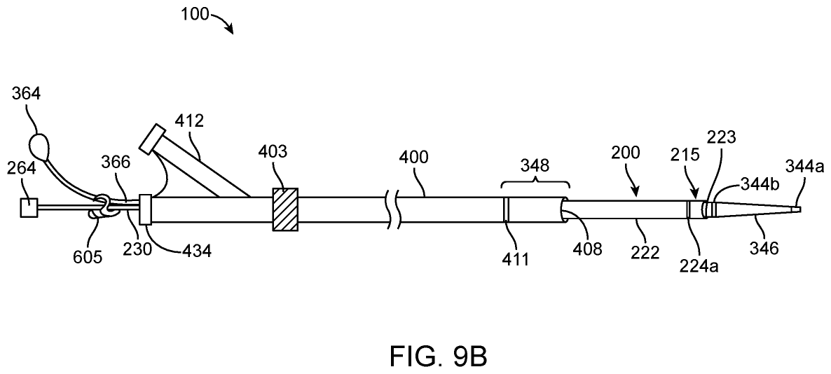

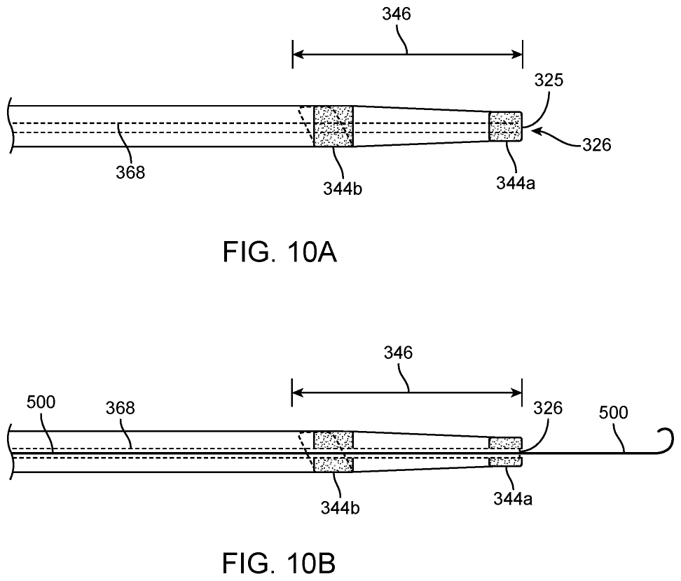

FIGS. 9A-9B illustrate an implementation of a distal access system 100 including devices for accessing and treating an intracranial or cerebral aneurysm, such as by deploying a flow diverter. FIG. 9A is an exploded view of an implementation of an access catheter system and FIG. 9B is an assembled view of the catheter system of FIG. 9A. FIG. 10A is a detailed view of the catheter advancement element 300 of FIG. 9A taken along circle C-C. FIG. 10B is a detailed view of a catheter advancement element having a guidewire 500 in the lumen 368 so that the distal end of the guidewire 500 extends distal to the distal opening 326 of the lumen 368. The distal access system 100 is capable of providing quick and simple access to distal target anatomy, particularly the tortuous anatomy of the intracranial and cerebral vasculature. The system 100 can be a single operator system such that each of the components and systems can be delivered and used together by one operator through a single point of manipulation requiring minimal hand movements. As will be described in more detail below, all wire and catheter manipulations can occur at or in close proximity to a single rotating hemostatic valve (RHV) or more than a single RHV co-located in the same device.

The system 100 can include one or more catheter systems 150, each having a catheter 200 and a catheter advancement element 300. The catheter system 150 is configured to be advanced through an access guide sheath 400. The catheter 200 is configured to be received through the guide sheath 400 and is designed to have exceptional deliverability. The catheter 200 can, but need not, be a distal access catheter having a distal tubular component coupled to a smaller outer diameter proximal control element. The distal tubular component being co-axial with a lumen of the guide sheath 400 provides a step-up in inner diameter within the conduit. The catheter need not include the proximal control element and instead can be a conventional, full-length catheter having a uniform diameter.

The catheter 200 can be delivered using a catheter advancement element 300 inserted through a lumen 223 of the catheter 200. The flexibility and deliverability of the distal access catheter 200 allow the catheter 200 to take the shape of the tortuous anatomy and avoids exerting straightening forces creating new anatomy. The distal access catheter 200 is capable of this even in the presence of the catheter advancement element 300 extending through its lumen. Thus, the flexibility and deliverability of the catheter advancement element 300 is on par or better than the flexibility and deliverability of the distal luminal portion 222 of the distal access catheter 200 in that both are configured to reach the middle cerebral artery (MCA) circulation without straightening out the curves of the anatomy along the way.

Still with respect to FIGS. 9A-9B, the distal access system 100 can include an access guide sheath 400 having a body 402 through which a working lumen extends from a proximal hemostasis valve 434 coupled to a proximal end region 403 of the body 402 to a distal opening 408 of a distal end region. The working lumen is configured to receive the catheter 200 therethrough such that a distal end of the catheter 200 can extend beyond a distal end of the sheath 400 through the distal opening 408. The guide sheath 400 can be used to deliver the catheters described herein as well as any of a variety of working devices known in the art. For example, the working devices can be configured to provide thrombotic treatments and can include large-bore catheters for delivery of flow diverters.

The sheath body 402 can extend from a proximal furcation or rotating hemostatic valve (RHV) 434 at a proximal end region 403 to a distal end opening 408 of the body 402. The proximal RHV 434 may include one or more lumens 412 molded into a connector body to connect to the working lumen of the body 402 of the guide sheath 400. The working lumen can receive the catheter 200 and/or any of a variety of working devices for delivery to a target anatomy. The RHV 434 can be constructed of thick-walled polymer tubing or reinforced polymer tubing. The RHV 434 allows for the introduction of devices through the guide sheath 400 into the vasculature, while preventing or minimizing blood loss and preventing air introduction into the guide sheath 400. The RHV 434 can be integral to the guide sheath 400 or the guide sheath 400 can terminate on a proximal end in a female Luer adaptor to which a separate hemostasis valve component, such as a passive seal valve, a Tuohy-Borst valve or RHV may be attached. The RHV 434 can have an adjustable opening that is open large enough to allow removal of devices that have adherent clot on the distal end opening 408 without causing the clot to dislodge at the RHV 434 during removal. Alternately, the RHV 434 can be removable, such as when a device is being removed from the sheath 400, to prevent clot dislodgement at the RHV 434. The RHV 434 can be a dual RHV or a multi-head RHV.

Contrast agent can be injected through the guide sheath 400 into the vessel to visualize the occlusion site by angiogram. For example, the guide sheath 400 can be positioned so that at least a portion is positioned within the carotid artery. The contrast agent may be injected through the sheath 400 once positioned in this location. Contrast agent can also be injected through one or more catheters inserted through the guide sheath 400. A baseline angiogram can be obtained, for example in the anterior/posterior (AP) and/or lateral views, prior to device insertion to assess occlusion location by injection of contrast media through the sheath 400 with fluoroscopic visualization. Fluoroscopic visualization may continue as the catheter system is advanced and subsequent angiograms can be captured periodically to assess reperfusion. The baseline angiogram image can be superimposed, such as with digital subtraction angiography, so that the vasculature and/or occlusion site are visible while the catheter system is advanced.

Once the catheter system 150 is advanced into position (the positioning will be described in more detail below), the catheter advancement element 300 can be withdrawn and removed from the system. In some implementations, the catheter 200 can be used as a support catheter to deliver a stent or flow diverter to the treatment site (e.g., within the carotid or a cerebral artery) as will be described elsewhere herein.

In an implementation, the guide sheath 400 includes one or more radiopaque markers 411. The radiopaque markers 411 can be disposed near the distal end opening 408. For example, a pair of radiopaque bands may be provided. The radiopaque markers 411 or markers of any of the system components can be swaged, painted, embedded, or otherwise disposed in or on the body. In some implementations, the radiopaque markers include a barium polymer, tungsten polymer blend, tungsten-filled or platinum-filled marker that maintains flexibility of the devices and improves transition along the length of the component and its resistance to kinking. In some implementations, the radiopaque markers are a tungsten-loaded PEBAX or polyurethane that is heat welded to the component.

The guide sheath markers 411 are shown in the figures as rings around a circumference of one or more regions of the body 402. However, the markers 411 can have other shapes or create a variety of patterns that provide orientation to an operator regarding the position of the distal opening 408 within the vessel. Accordingly, an operator may visualize a location of the distal opening 408 under fluoroscopy to confirm that the distal opening 408 is directed toward a target anatomy where a catheter 200 is to be delivered. For example, radiopaque marker(s) 411 allow an operator to rotate the body 402 of the guide sheath 400 at an anatomical access point, e.g., a groin of a patient, such that the distal opening provides access to an ICA by subsequent working device(s), e.g., catheters and wires advanced to the ICA. In some implementations, the radiopaque marker(s) 411 include platinum, gold, tantalum, tungsten or any other substance visible under an x-ray fluoroscope. Any of the various components of the systems described herein can incorporate radiopaque markers.

Still with respect to FIGS. 9A-9B, the catheter 200 can include a relatively flexible, distal luminal portion 222 coupled to a stiffer, kink-resistant proximal extension or proximal control element 230. The term “control element” as used herein can refer to a proximal region configured for a user to cause pushing movement in a distal direction as well as pulling movement in a proximal direction. The control elements described herein may also be referred to as spines, tethers, push wires, push tubes, or other elements having any of a variety of configurations. The proximal control element 230 can be a hollow or tubular element. The proximal control element 230 can also be solid and have no inner lumen, such as a solid rod, ribbon or other solid wire type element. Generally, the proximal control elements described herein are configured to move its respective component (to which it may be attached or integral) in a bidirectional manner through a lumen.

A single, inner lumen 223 extends through the luminal portion 222 between a proximal end and a distal end of the luminal portion 222 (the lumen 223 is visible in FIG. 9B). In some implementations, a proximal opening 242 into the lumen 223 can be located near where the proximal control element 230 couples with the distal luminal portion 222. In other implementations, the proximal opening 242 into the lumen 223 is at a proximal end region of the catheter 200. A distal opening 231 from the lumen 223 can be located near or at the distal-most end 215 of the luminal portion 222. The inner lumen 223 of the catheter 200 can have a first inner diameter and the working lumen of the guide sheath 400 can have a second, larger inner diameter. Upon insertion of the catheter 200 through the working lumen of the sheath 400, the lumen 223 of the catheter 200 can be configured to be fluidly connected and contiguous with the working lumen of the sheath 400 such that fluid flow into and/or out of the system 100 is possible, such as by applying suction from a vacuum source coupled to the system 100 at a proximal end. The combination of sheath 400 and catheter 200 can be continuously in communication with the bloodstream at the proximal end with advancement and withdrawal of catheter 200.

The distal luminal portion 222 of the catheter 200 can have one or more radiopaque markings 224. A first radiopaque marker 224a can be located near the distal-most end 215 to aid in navigation and proper positioning of the distal-most end 215 under fluoroscopy. Additionally, a proximal region of the catheter 200 may have one or more proximal radiopaque markers 224b so that the overlap region 348 can be visualized as the relationship between a radiopaque marker 411 on the guide sheath 400 and the radiopaque marker 224b on the catheter 200. The proximal region of the catheter 200 may also have one or more radiopaque markings providing visualization, for example, near the proximal opening 242 into the single lumen 223 of the catheter 200 as will be described in more detail below. In an implementation, the two radiopaque markers (marker 224a near the distal-most end 215 and a more proximal marker 224b) are distinct to minimize confusion of the fluoroscopic image, for example the catheter proximal marker 224b may be a single band and the marker 411 on the guide sheath 400 may be a double band and any markers on a working device delivered through the distal access system can have another type of band or mark. The radiopaque markers 224 of the distal luminal portion 222, particularly those near the distal end region navigating extremely tortuous anatomy, can be relatively flexible such that they do not affect the overall flexibility of the distal luminal portion 222 near the distal end region. The radiopaque markers 224 can be tungsten-loaded or platinum-loaded markers that are relatively flexible compared to other types of radiopaque markers used in devices where flexibility is not paramount. In some implementations, the radiopaque marker can be a band of tungsten-loaded PEBAX having a durometer of Shore A 35D.

The proximal control element 230 can include one or more markers 232 to indicate the overlap between the distal luminal portion 222 of the catheter 200 and the sheath body 402 as well as the overlap between the distal luminal portion 222 of the catheter 200 and other interventional devices that may extend through the distal luminal portion 222. At least a first mark can be an RHV proximity marker positioned so that when the mark is aligned with the sheath proximal hemostasis valve 434 during insertion of the catheter 200 through the guide sheath 400, the catheter 200 is positioned at the distal-most position with the minimal overlap length needed to create the seal between the catheter 200 and the working lumen. At least a second marker 232 can be a Fluoro-saver marker that can be positioned on the control element 230 and located a distance away from the distal-most end 215 of the distal luminal portion 222. In some implementations, a marker 232 can be positioned about 100 cm away from the distal-most end 215 of the distal luminal portion 222. The markers 232 can be positioned on the catheter so that one or more markers are visible to an operator outside the patient (and outside the guide sheath 400) during use. One or more markers can also be visible to an operator inside the patient (and inside the guide sheath 400 or beyond a distal end of the guide sheath 400) during use such that they are visualized under fluoroscopy.

The catheter 200 shown in FIGS. 9A-9B is less than full-length and includes a rapid exchange opening 242 into the distal luminal portion 222. The catheter 200 can also be a full-length catheter having a lumen that ends between the distal and proximal opening, the proximal opening configured to remain outside the sheath hub and outside the patient.

Still with respect to FIGS. 9A-9B and also FIG. 10A, the catheter advancement element 300 can include a non-expandable, flexible elongate body 360 and a proximal portion 366. The catheter advancement element 300 and the catheter 200 described herein may be configured for rapid exchange or over-the-wire methods. For example, the flexible elongate body 360 can be a tubular portion extending the entire length of the catheter advancement element 300 and can have a proximal opening from the lumen 368 of the flexible elongate body 360 that is configured to extend outside the patient's body during use. Alternatively, the tubular portion can have a proximal opening positioned such that the proximal opening remains inside the patient's body during use. The proximal portion 366 can be a proximal element coupled to a distal tubular portion 360 and extending proximally therefrom. A proximal opening from the tubular portion 360 can be positioned near where the proximal element 366 couples to the tubular portion 360. Alternatively, the proximal portion 366 can be an extension of the tubular portion 360 having a length that extends to a proximal opening near a proximal terminus of the catheter advancement element 300 (i.e. outside a patient's body). A luer 364 can be coupled to the proximal portion 366 at the proximal end region so that tools, such as a guidewire, can be advanced through the lumen 368 of the catheter advancement element 300. A syringe or other component can be coupled to the luer 364 in order to draw a vacuum and/or inject fluids through the lumen 368. The syringe coupled to the luer 364 can also be used to close off the lumen of the catheter advancement element 300 to maximize the piston effect described elsewhere herein.

The configuration of the proximal portion 366 can vary. In some implementations, the proximal portion 366 is simply a proximal extension of the flexible elongate body 360 that does not change significantly in structure but changes in flexibility. For example, the proximal portion 366 transitions from the very flexible distal regions of the catheter advancement element 300 towards less flexible proximal regions of the catheter advancement element 300. In some implementations, the proximal portion 366 can provide a relatively stiff proximal end suitable for manipulating (e.g., advancing and withdrawing) the more distal regions of the catheter advancement element 300 relative to the anatomy and/or the outer catheter 200. The proximal portion 366 can be formed of a less flexible polymer than the flexible elongate body. The proximal portion 366 can be fully polymeric having no reinforcement or the proximal portion 366 can be a reinforced polymer portion. The configuration of the proximal portion 366 can vary depending on whether the catheter advancement element 300 is to be used with a full-length catheter or a catheter having only a short distal tubular portion. The catheter advancement element 300 used with a full-length catheter need not rely upon a proximal reinforcement in order to advance the catheter system through the anatomy and can instead rely on the proximal stiffness of the outer catheter. A catheter advancement element 300 used with a partial tube outer catheter may benefit from a stiffer reinforcement within its proximal end region for advancing the system.

In some implementations, the proximal portion 366 is a metal reinforced segment. The metal reinforced segment can be positioned a distance away from the distal end of the elongate body. For example, the metal reinforced segment can be about 50 cm from the distal end. The metal reinforced segment can have an inner diameter of about 0.021″ and an outer diameter of about 0.027″. The metal reinforced segment can be a spine. The metal reinforced segment can be a hypotube. In other implementations, the proximal portion 366 is a hypotube. The hypotube may be exposed or may be coated by a polymer. In still further implementations, the proximal portion 366 may be a tubular polymer portion reinforced by a coiled ribbon or braid. The proximal portion 366 can have the same outer diameter as the flexible elongate body or can have a smaller outer diameter as the flexible elongate body.

The proximal portion 366 need not include a lumen. For example, the proximal portion 366 can be a solid rod, ribbon, or wire have no lumen extending through it that couples to the tubular elongate body 360. Where the proximal portion 366 is described herein as having a lumen, it should be appreciated that the proximal portion 366 can also be solid and have no lumen. The proximal portion 366 is generally less flexible than the elongate body 360 and can transition to be even more stiff towards the proximal-most end of the proximal portion 366. Thus, the catheter advancement element 300 can have an extremely soft and flexible distal end region 346 that transitions proximally to a stiff proximal portion 366 well suited for pushing and/or torqueing the distal elongate body 360.

The elongate body 360 can be received within and extended through the internal lumen 223 of the distal luminal portion 222 of the catheter 200 (see FIG. 2B). The elongate body 360 or tubular portion can have an outer diameter. The outer diameter of the tubular portion can have at least one snug point. The at least one snug point provides a close fit between the elongate body 360 and the distal luminal portion 222 that minimizes a distal lip or edge at the distal end of the catheter 200, but that still allows for movement relative to one another so as to allow a user to achieve a desired extension or withdrawal of the catheter advancement element 300 relative to the catheter 200 or the catheter 200 relative to the catheter advancement element 300. The snug point allows for movement between the catheters upon application of a relatively small load so as to avoid any negative impact on usability within a patient. A difference between the inner diameter of the catheter 200 and the outer diameter of the tubular portion at the snug point can be no more than about 0.015″ (0.381 mm), or can be no more than about 0.010″ (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm).

As will be described in more detail below, the catheter advancement element 300 can also include a distal end region 346 located distal to the at least one snug point of the tubular portion. The distal end region 346 can have a length and taper along at least a portion of the length. The distal end region 346 of the catheter advancement element 300 can be extended beyond the distal end of the catheter 200 as shown in FIG. 9B. The proximal portion 366 of the catheter advancement element 300 or proximal extension is coupled to a proximal end region of the elongate body 360 and extends proximally therefrom. The proximal portion 366 can be less flexible than the elongate body 360 and configured for bi-directional movement of the elongate body 360 of the catheter advancement element 300 within the luminal portion 222 of the catheter 200, as well as for movement of the catheter system 100 as a whole. The elongate body 360 can be inserted in a coaxial fashion through the internal lumen 223 of the luminal portion 222. The outer diameter of at least a region of the elongate body 360 can be sized to substantially fill at least a portion of the internal lumen 223 of the luminal portion 222.

The overall length of the catheter advancement element 300 (e.g. between the proximal end through to the distal-most tip) can vary, but generally is long enough to extend through the support catheter 200 plus at least a distance beyond the distal end of the support catheter 200 while at least a length of the proximal portion 366 remains outside the proximal end of the guide sheath 400 and outside the body of the patient. In some implementations, the overall length of the catheter advancement element 300 is about 145 to about 150 cm and has a working length of about 140 cm to about 145 cm from a proximal tab or hub to the distal-most end 325. The elongate body 360 can have a length that is at least as long as the luminal portion 222 of the catheter 200 although the elongate body 360 can be shorter than the luminal portion 222 so long as at least a minimum length remains inside the luminal portion 222 when a distal portion of the elongate body 360 is extended distal to the distal end of the luminal portion 222 to form a snug point or snug region with the catheter. In some implementations, this minimum length of the elongate body 360 that remains inside the luminal portion 222 when the distal end region 346 is positioned at its optimal advancement configuration is at least about 5 cm, at least about 6 cm, at least about 7 cm, at least about 8 cm, at least about 9 cm, at least about 10 cm, at least about 11 cm, or at least about 12 cm up to about 50 cm. In some implementations, the shaft length of the distal luminal portion 222 can be about 35 cm up to about 75 cm and shorter than a working length of the guide sheath and the insert length of the elongate body 360 can be at least about 45 cm, 46 cm, 47 cm, 48 cm, 48.5 cm, 49 cm, 49.5 cm up to about 85 cm.

The length of the elongate body 360 can allow for the distal end of the elongate body 360 to reach cerebrovascular targets or occlusions within, for example, segments of the internal carotid artery including the cervical (C1), petrous (C2), lacerum (C3), cavernous (C4), clinoid (C5), ophthalmic (C6), and communicating (C7) segments of the internal carotid artery (ICA) as well as branches off these segments including the M1 or M2 segments of the middle cerebral artery (MCA), anterior cerebral artery (ACA), anterior temporal branch (ATB), and/or posterior cerebral artery (PCA). The distal end region of the elongate body 360 can reach these distal target locations while the proximal end region of the elongate body 360 remains proximal to or below the level of severe turns along the path of insertion. For example, the entry location of the catheter system can be in the femoral artery and the target occlusion location can be distal to the right common carotid artery, such as within the M1 segment of the middle cerebral artery on the right side. The proximal end region of the elongate body 360 where it transitions to the proximal portion 366 can remain within a vessel that is proximal to severely tortuous anatomy, such as the carotid siphon, the right common carotid artery, the brachiocephalic trunk, the take-off into the brachiocephalic artery from the aortic arch, the aortic arch as it transitions from the descending aorta. This avoids inserting the stiffer proximal portion 366, or the material transition between the stiffer proximal portion 366 and the elongate body 360, from taking the turn of the aortic arch or the turn of the brachiocephalic take-off from the aortic arch, which both can be very severe. The lengths described herein for the distal luminal portion 222 also can apply to the elongate body 360 of the catheter advancement element.

The proximal portion 366 can have a length that varies as well. In some implementations, the proximal portion 366 is about 90 cm up to about 95 cm. The distal portion extending distal to the distal end of the luminal portion 222 can include distal end region 346 that protrudes a length beyond the distal end of the luminal portion 222 during use of the catheter advancement element 300. The distal end region 346 of the elongate body 360 that is configured to protrude distally from the distal end of the luminal portion 222 during advancement of the catheter 200 through the tortuous anatomy of the cerebral vessels, as will be described in more detail below. The proximal portion 366 coupled to and extending proximally from the elongate body 360 can align generally side-by-side with the proximal control element 230 of the catheter 200. The arrangement between the elongate body 360 and the luminal portion 222 can be maintained during advancement of the catheter 200 through the tortuous anatomy to reach the target location for treatment in the distal vessels and aids in preventing the distal end of the catheter 200 from catching on tortuous branching vessels, as will be described in more detail below.

In some implementations, the elongate body 360 can have a region of relatively uniform outer diameter extending along at least a portion of its length and the distal end region 346 tapers down from the uniform outer diameter. The outer diameter of the elongate body 360 also can taper or step down in outer diameter proximally, for example near where the elongate body 360 couples or transitions to the proximal portion 366. The outer diameter of the elongate body 360 need not change in outer diameter near where the elongate body 360 couples or transitions to the proximal portion 366. In some implementations, the region of relatively uniform outer diameter can extend along a majority of the working length of the catheter advancement element 300 including the proximal portion 366. This first region of uniform outer diameter can transition to a second region of uniform outer diameter located distal to the first region. The transition can incorporate a smooth taper or step change in outer diameter between the two regions. The second region of uniform outer diameter having the larger size and located distal to the first region can be useful in filling a lumen of a larger bore catheter without the entire working length of the elongate body needing to have this larger size. In this implementation, the elongate body 360 can have a distal taper changing in diameter from the second uniform diameter region towards the distal opening and a proximal taper changing in diameter from the second uniform diameter region towards the first region of uniform outer diameter.

Depending upon the inner diameter of the catheter 200, the difference between the inner diameter of catheter 200 and the outer diameter of the elongate body 360 along at least a portion of its length, such as at least 10 cm of its length, preferably at least 15 cm of its length can be no more than about 0.015″ (0.381 mm), such as within a range of about 0.003″-0.015″ (0.0762 mm-0.381 mm) or between 0.006″-0.010″ (0.1524 mm-0.254 mm). Thus, the clearance between the catheter 200 and the elongate body 360 can result in a space on opposite sides that is no more than about 0.008″ (0.2032 mm), or can be no more than about 0.005″ (0.127 mm), for example, from about 0.001″ up to about 0.006″ (0.0254 mm-0.1524 mm), preferably about 0.002″ to about 0.005″ (0.0508 mm-0.127 mm), and more preferably about 0.003″ to about 0.005″ (0.0762 mm-0.0508 mm).

The catheter advancement element 300 has a large outer diameter and a relatively small inner diameter, particularly when a guidewire extends into or through the lumen of the catheter advancement element 300. The elongate body 360 can have an overall shape profile from proximal end to distal end that transitions from a first outer diameter having a first length to a tapering outer diameter having a second length. The first length of this first outer diameter region (i.e. the snug-fitting region between the distal luminal portion 222 and the elongate body 360) can be at least about 5 cm, or 10 cm, up to about 50 cm. In other implementations, the snug-fitting region can extend from the proximal tab or luer 364 substantially to the tapered distal end region 346 which depending on the length of the catheter advancement element 300, can be up to about 170 cm. The length of the tapering outer diameter of the distal end region 346 can be about 0.5 cm to about 5 cm, about 1 cm to about 4 cm, or about 1.5 cm to about 3 cm, or between 2.0 cm and about 2.5 cm. In some implementations, the length of the distal end region 346 varies depending on the inner diameter of the catheter 200 with which the catheter advancement element 300 is to be used. For example, the length of the distal end region 346 can be shorter (e.g. 1.2 cm) for a catheter advancement element 300 sized to be used with a catheter 200 having an inner diameter of about 0.054″ (1.372 mm) and can be longer (e.g. 2.5 cm) for a catheter advancement element 300 sized to be used with a catheter 200 having an inner diameter of about 0.088″ (2.235 mm). The distal end region 346 can be a constant taper from the larger outer diameter of the elongate body 360 (e.g. the distal end of the marker 344b) down to a second smaller outer diameter at the distal-most terminus (e.g. the proximal end of the marker 344a) as shown in FIG. 10A. In some implementations, the constant taper of the distal end region 346 can be from about 0.048″ outer diameter down to about 0.031″ (0.787 mm) outer diameter over a length of about 1 cm. In some implementations, the constant taper of the distal end region 346 can be from 0.062″ (1.575 mm) outer diameter to about 0.031″ (0.787 mm) outer diameter over a length of about 2 cm. In still further implementations, the constant taper of the distal end region 346 can be from 0.080″ (2.032 mm) outer diameter to about 0.031″ (0.787 mm) outer diameter over a length of about 2.5 cm. The length of the constant taper of the distal end region 346 can vary, for example, between 0.8 cm to about 2.5 cm, or between 1 cm and 3 cm, or between 2.0 cm and 2.5 cm. The angle of the taper can vary depending on the outer diameter of the elongate body 360. For example, the angle of the taper can be between 0.9 to 1.6 degrees relative to horizontal. The angle of the taper can be between 2-3 degrees from a center line of the elongate body 360. The length of the taper of the distal end region 346 can be between about 5 mm to 20 mm or about 20 mm to about 50 mm.

The distal end region 346 of the elongate body 360 can also be shaped with or without a taper. When the catheter advancement element 300 is inserted through the catheter 200, this distal end region 346 is configured to extend beyond and protrude out through the distal-most end 215 of the luminal portion 222 whereas the more proximal region of the body 360 (i.e. the first length described above) remains within the luminal portion 222.

As mentioned, the distal-most end 215 of the luminal portion 222 can be blunt and have no change in the dimension of the outer diameter whereas the distal end region 346 can be tapered providing an overall elongated tapered geometry of the catheter system. The outer diameter of the elongate body 360 also approaches the inner diameter of the luminal portion 222 such that the step-up from the elongate body 360 to the outer diameter of the luminal portion 222 is minimized. Minimizing this step-up prevents issues with the lip formed by the distal end of the luminal portion 222 catching on the tortuous neurovasculature, such as around the carotid siphon near the ophthalmic artery branch, when the distal end region 346 in combination with the distal end region of the catheter 200 bends and curves along within the vascular anatomy. In some implementations, the inner diameter of the luminal portion 222 can be at least about 0.052″ (1.321 mm), about 0.054″ (1.372 mm) and the maximum outer diameter of the elongate body 360 can be about 0.048″ (1.219 mm) such that the difference between them is about 0.006″ (0.1524 mm). In some implementations, the inner diameter of the luminal portion 222 can be about 0.070″ (1.778 mm) and the maximum outer diameter of the elongate body 360 can be about 0.062″ (1.575 mm) such that the difference between them is about 0.008″ (0.2032 mm). In some implementations, the inner diameter of the luminal portion 222 can be about 0.088″ (2.235 mm) and the maximum outer diameter of the elongate body 360 can be about 0.080″ (2.032 mm) such that the difference between them is about 0.008″ (0.2032 mm). In some implementations, the inner diameter of the luminal portion 222 can be about 0.072″ (1.829 mm) and the maximum outer diameter of the elongate body 360 is about 0.070″ (1.778 mm) such that the difference between them is only 2 thousandths of an inch (0.002″/0.0508 mm). In other implementations, the maximum outer diameter of the elongate body 360 is about 0.062″ (1.575 mm) such that the difference between them is about 0.010″ (0.254 mm). Despite the outer diameter of the elongate body 360 extending through the lumen of the luminal portion 222, the luminal portion 222 and the elongate body 360 extending through it in co-axial fashion are flexible enough to navigate the tortuous anatomy leading to the level of M1 or M2 arteries without kinking and without damaging the vessel. It is preferred to deliver a catheter that is as large in inner diameter for the passage of larger-sized flow diverter delivery systems.

The dimensions provided herein are approximate and each dimensions may have an engineering tolerance or a permissible limit of variation. Use of the term “about,” “approximately,” or “substantially” are intended to provide such permissible tolerance to the dimension being referred to. Where “about” or “approximately” or “substantially” is not used with a particular dimension herein that that dimension need not be exact.

The elongate body 360 of the catheter advancement element 300 can have a lumen 368 with an inner diameter that does not change over the length of the elongate body even in the presence of the tapering of the distal end region 346. Thus, the inner diameter of the lumen 368 extending through the tubular portion of the catheter advancement element 300 can remain uniform and the wall thickness of the distal end region 346 can decrease to provide the taper. The wall thickness can thin distally along the length of the taper. Thus, the material properties in combination with wall thickness, angle, length of the taper can all contribute to the overall maximum flexibility of the distal-most end of the distal end region 346. The catheter advancement element 300 undergoes a transition in flexibility from the distal-most end towards the snug point where it achieves an outer diameter that is no more than about 0.010″ (0.254 mm) different from the inner diameter of the catheter 200.

The inner diameter of the elongate body 360 can be constant along its length even where the single lumen passes through the tapering distal end region 346. Alternatively, the inner diameter of the elongate body 360 can have a first size through the tapering distal end region 346 and a second, larger size through the cylindrical section of the elongate body 360. The cylindrical section of the elongate body 360 can have a constant wall thickness or a wall thickness that varies to a change in inner diameter of the cylindrical section. As an example, the outer diameter of the cylindrical section of the elongate body 360 can be about 0.080″. The inner diameter of the elongate body 360 within the cylindrical section can be uniform along the length of the cylindrical section and can be about 0.019″. The wall thickness in this section, in turn, can be about 0.061″. As another example, the outer diameter of the cylindrical section of the elongate body 360 can again be between about 0.080″. The inner diameter of the elongate body 360 within the cylindrical section can be non-uniform along the length of the cylindrical section and can step-up from a first inner diameter of about 0.019″ to a larger second inner diameter of about 0.021″. The wall thickness, in turn, can be about 0.061″ at the first inner diameter region and about 0.059″ at the second inner diameter region. The wall thickness of the cylindrical portion of the elongate body 360 can be between about 0.050″ to about 0.065″. The wall thickness of the tapered distal end region 346 near the location of the proximal marker band can be the same as the cylindrical portion (between about 0.050″ and about 0.065″) and become thinner towards the location of the distal marker band. As an example, the inner diameter at the distal opening from the single lumen can be about 0.020″ and the outer diameter at the distal opening (i.e. the outer diameter of the distal marker band) and be about 0.030″ resulting in a wall thickness of about 0.010″ compared to the wall thickness of the cylindrical portion that can be up to about 0.065″. Thus, the outer diameter of the distal tip 346 can taper as can the wall thickness. A wall thickness of the intermediate segment and an un-tapered portion of the tip segment can be about 0.050″ to about 0.065″. The wall thickness of the intermediate segment and the un-tapered portion can be constant. The inner diameter of the intermediate segment and the tapered end region can be constant.

A tip segment of the flexible elongate body can have a tapered portion that tapers distally from a first outer diameter to a second outer diameter. The second outer diameter can be about ½ of the first outer diameter. The second outer diameter can be about 40% of the first outer diameter. The second outer diameter can be about 65% of the first outer diameter. The first outer diameter can be about 0.062″ up to about 0.080″. The second outer diameter can be about 0.031″. The second outer diameter can be about 50% of the first outer diameter, about 40% of the first outer diameter, or about 65% of the first outer diameter.

The length of the taper can also vary depending on the anatomy of the target region. The distal end region 346 can achieve its soft, atraumatic and flexible characteristic due to a material property other than due to a change in outer dimension to facilitate endovascular navigation to an occlusion in tortuous anatomy. Additionally or alternatively, the distal end region 346 of the elongate body 360 can have a transition in flexibility along its length. The most flexible region of the distal end region 346 can be its distal terminus. Moving along the length of the distal end region 346 from the distal terminus towards a region proximal to the distal terminus. For example, the distal end region 346 can be formed of a material having a Shore material hardness of no more than 35D or about 62A and transitions proximally to be less flexible near where it is formed of a material having a material hardness of no more than 55D and 72D up to the proximal portion 366, which can be a stainless steel hypotube, or a combination of a material property and tapered shape. The materials used to form the regions of the elongate body 360 can include PEBAX (such as PEBAX 25D, 35D, 55D, 69D, 72D) or a blend of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer), with a lubricious additive compound, such as Mobilize (Compounding Solutions, Lewiston, Maine). In some implementations, the material used to form a region of the elongate body 360 can be Tecothane 62A. Incorporation of a lubricious additive directly into the polymer elongate body means incorporation of a separate lubricious liner, such as a Teflon liner, is unnecessary. This allows for a more flexible element that can navigate the distal cerebral anatomy and is less likely to kink. Similar materials can be used for forming the distal luminal portion 222 of the catheter 200 providing similar advantages. The flexibility of the distal end region 346 can be achieved by a combination of flexible lubricious materials and tapered shapes. For example, the length of the distal end region 346 can be kept shorter than 2 cm-3 cm, but maintain optimum deliverability due to a change in flexible material from distal-most end 325 towards a more proximal region a distance away from the distal-most end 325. In an implementation, the elongate body 360 is formed of PEBAX (polyether block amide) embedded silicone designed to maintain the highest degree of flexibility. The wall thickness of the distal end of the luminal portion 222 can also be made thin enough such that the lip formed by the distal end of the luminal portion 222 relative to the elongate body 360 is minimized.

The elongate body 360 has a benefit over a microcatheter in that it can have a relatively large outer diameter that is just 0.003″-0.010″ (0.0762 mm-0.254 mm) smaller than the inner diameter of the distal luminal portion 222 of the catheter 200 and still maintain a high degree of flexibility for navigating tortuous anatomy. When the gap between the two components is too tight (e.g. less than about 0.003″ (0.0762 mm), the force needed to slide the catheter advancement element 300 relative to the catheter 200 can result in damage to one or both of the components and increases risk to the patient during the procedure. The gap results in too tight of a fit to provide optimum relative sliding. When the gap between the two components is too loose (e.g. greater than about 0.010″/0.254 mm), the distal end of the catheter 200 forms a lip that is prone to catch on carotid dissections or branching vessels during advancement through tortuous neurovasculature, such as around the carotid siphon where the ophthalmic artery branches off and the piston effect of withdrawal of the elongate body 360 can be decreased or lost.

The gap in ID/OD between the elongate body 360 and the distal luminal portion 222 can be in this size range (e.g. 0.003″-0.015″ (0.0762 mm-0.381 mm) or between 0.006″-0.010″ (0.152 mm-0.254 mm)) along a majority of their lengths. For example, the elongate body 360 can have a relatively uniform outer diameter that is between about 0.048″ (1.219 mm) to about 0.080″ (2.032 mm) from a proximal end region to a distal end region up to a point where the taper of the distal end region 346 begins. Similarly, the distal luminal portion 222 of the catheter 200 can have a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm-0.381 mm). The distal end region 346 of the elongate body 360 that is tapered will have a larger gap size relative to the inner diameter of the distal luminal portion 222. During use, however, this tapered distal end region 346 is configured to extend distal to the distal end of the catheter 200 such that the region of the elongate body 360 having an outer diameter sized to match the inner diameter of the distal luminal portion 222 is positioned within the lumen of the catheter 200 such that it can minimize the lip at the distal end of the catheter 200.

The elongate body 360 can be formed of various materials that provide a suitable flexibility and lubricity. Example materials include high density polyethylene, 77A PEBAX, 33D PEBAX, 42D PEBAX, 46D PEBAX, 54D PEBAX, 69D PEBAX, 72D PEBAX, 90D PEBAX, and mixtures thereof or equivalent stiffness and lubricity material. In some implementations, the elongate body 360 is an unreinforced, non-torqueing catheter having a relatively large outer diameter designed to fill the lumen it is inserted through and a relatively small inner diameter to minimize any gaps at a distal-facing end of the device. In other implementations, at least a portion of the elongate body 360 can be reinforced to improve navigation and torqueing (e.g. braided reinforcement layer). The flexibility of the elongate body 360 can increase towards the distal end region 346 such that the distal region of the elongate body 360 is softer, more flexible, and articulates and bends more easily than a more proximal region. For example, a more proximal region of the elongate body can have a bending stiffness that is flexible enough to navigate tortuous anatomy, such as the carotid siphon, without kinking. If the elongate body 360 has a braid reinforcement layer along at least a portion of its length, the braid reinforcement layer can terminate a distance proximal to the distal end region 346. For example, the distance from the end of the braid to the distal-most end 325 can be about 10 cm to about 15 cm or from about 4 cm to about 10 cm or from about 4 cm up to about 15 cm.

In some implementations, the elongate body 360 can be generally tubular along at least a portion of its length such that it has a single lumen 368 extending parallel to a longitudinal axis of the catheter advancement element 300 (see FIGS. 9A-9B and 10A-10B). In an implementation, the single lumen 368 of the elongate body 360 is sized to accommodate a guidewire, however use of the catheter advancement element 300 generally eliminates the need for a guidewire lead. Preferably, the assembled system includes no guidewire or a guidewire parked inside the lumen 368 retracted away from the distal opening. Guidewires are designed to be exceptionally flexible so that they deflect to navigate the severe turns of the anatomy. However, many workhorse guidewires have a stiffness along their longitudinal axis and/or are small enough in outer diameter that they find their own paths through an occlusion rather than slipping around the occlusion or get hung up on vessel wall dissections increasing the risk of perforations. In some cases, these guidewires can cause perforations and/or dissections of the vessel itself. Guidewires tend to get redirected into branches rather than remaining within the larger vessel. This makes them helpful for selecting a branch, but problematic for navigating tortuous anatomy and following the main flow of blood. Thus, even though the guidewire may have an outer diameter at its distal tip region that is small and very flexible at the distal tip, guidewires typically are incapable of atraumatically probing an occlusion or other structure such that the pose a risk of perforation with repeated advancement. Guidewires do not deflect upon encountering something relatively dense, such as the proximal face of the occlusion or a dissection flap. Instead, guidewires embed and penetrate such structures. The catheter advancement element 300 has a softness, taper, and sizing that finds and/or creates space. For example, the catheter advancement element 300 upon encountering an occlusion, such as an atherosclerotic lesion or embolus, can slide between a portion of the occlusion and the vessel wall rather than penetrating through it like a guidewire does. In the case of a partially occluded vessel, such as a narrowing within the carotid artery, the catheter advancement element 300 can atraumatically and safely find the path through the narrowing. The catheter advancement element 300 also deflects away from a dissection flap so as to remain within the larger lumen. The softness, taper, and sizing of the catheter advancement element 300 allows for it to be repeatedly passed through the carotid and into the cerebral arteries without penetrating or taking a detour relative to these structures. The distal tip region deflects and passes by these structures so that the catheter system is advanced past them to a distal occlusion site or probes and wedges near them in a safe manner.

A guidewire 500 can extend through the single lumen 368 generally concentrically from a proximal opening to a distal opening 326 at the distal end 325 of the catheter advancement element 300 (see FIG. 10B). In some implementations, the proximal opening is at the proximal end of the catheter advancement element 300 such that the catheter advancement element 300 is configured for over-the-wire (OTW) methodologies. In other implementations, the proximal opening is a rapid exchange opening through a wall of the catheter advancement element 300 such that the catheter advancement element 300 is configured for rapid exchange rather than or in addition to OTW. In this implementation, the proximal opening extends through the sidewall of the elongate body 360 and is located a distance away from a proximal tab or luer 364 and distal to the proximal portion 366. The proximal opening can be located a distance of about 10 cm from the distal end region 346 up to about 20 cm from the distal end region 346. In some implementations, the proximal opening can be located near a region where the elongate body 360 is joined to the proximal portion 366, for example, just distal to an end of the hypotube. In other implementations, the proximal opening is located more distally, such as about 10 cm to about 18 cm from the distal-most end of the elongate body 360. A proximal opening that is located closer to the distal end region 346 allows for easier removal of the catheter advancement element 300 from the catheter 200 leaving the guidewire in place for a “rapid exchange” type of procedure. Rapid exchanges can rely on only a single person to perform the exchange. The catheter advancement element 300 can be readily substituted for another device using the same guidewire that remains in position. The single lumen 368 of the elongate body 360 can be configured to receive a guidewire 500 in the range of 0.014″ (0.356 mm) and 0.018″ (0.457 mm) diameter, or in the range of between 0.014″ and 0.022″ (0.356 mm-0.559 mm). In this implementation, the inner luminal diameter of the elongate body 360 can be between 0.020″ and 0.024″ (0.508 mm-0.610 mm). The guidewire, the catheter advancement element 300, and the catheter 200 can all be assembled co-axially for insertion through the working lumen of the guide sheath 400. The inner diameter of the lumen 368 of the elongate body 360 can be 0.019″ to about 0.021″ (0.483 mm-0.533 mm). The distal opening 326 from the lumen 368 can have an inner diameter that is between about 0.018″ to about 0.024″ (0.457 mm-0.610 mm). The distal opening 326 from the lumen 368 can have an inner diameter that is between about 0.016″ to about 0.028″. The distal opening 326 is sized to receive a guidewire that can be a 0.014″ to a 0.024″ guidewire.

The region near the distal end region 346 can be tapered such that the outer diameter tapers over a length of about 0.5 cm to about 5 cm, or 1 cm to about 4 cm, or other length as described elsewhere herein. The larger outer diameter can be at least about 1.5 times, 2 times, 2.5 times, or about 3 times larger than the smaller outer diameter. The distal end region 346 can taper along a distance from a first outer diameter to a second outer diameter, the first outer diameter being at least 1.5 times the second outer diameter. In some implementations, the distal end region 346 tapers from about 0.080″ (2.032 mm) to about 0.031″ (0.787 mm). In some implementations, the smaller outer diameter at a distal end of the taper can be about 0.026″ (0.66 mm) up to about 0.040″ (1.016 mm) and the larger outer diameter proximal to the taper is about 0.062″ (1.575 mm) up to about 0.080″ (2.032 mm). Also, the distal end region 346 can be formed of a material having a material hardness (e.g. 62A and 35D) that transitions proximally towards increasingly harder materials having (e.g. 55D and 72D) up to the proximal portion 366. A first segment of the elongate body 360 including the distal end region 346 can be formed of a material having a material hardness of 35D and a length of about 10 cm to about 12.5 cm. The first segment of the elongate body 360 including the distal end region 346 can be formed of a material having a material hardness of 62A and a length of about 10 cm to about 12.5 cm. A second segment of the elongate body 360 can be formed of a material having a material hardness of 55D and have a length of about 5 cm to about 8 cm. A third segment of the elongate body 360 can be formed of a material having a material hardness of 72D can be about 25 cm to about 35 cm in length. The three segments combined can form an insert length of the elongate body 360 from where the proximal portion 366 couples to the elongate body 360 to the terminus of the distal end region 346 that can be about 49 cm in length.

The catheter advancement element 300 can incorporate a reinforcement layer. The reinforcement layer can be a braid or other type of reinforcement to improve the torqueability of the catheter advancement element 300 and help to bridge the components of the catheter advancement element 300 having such differences in flexibility. The reinforcement layer can bridge the transition from the rigid, proximal portion 366 to the flexible elongate body 360. In some implementations, the reinforcement layer can be a braid positioned between inner and outer layers of PEBAX. The reinforcement layer can terminate a distance proximal to the distal end region 346. The distal end region 346 can be formed of a material having a material hardness of at most about 35D. The first segment can be unreinforced polymer having a length of about 4 cm up to about 12.5 cm without metal reinforcement. The third segment of the elongate body 360 located proximal to the first segment can include the reinforcement layer and can extend a total of about 37 cm up to the unreinforced distal segment. A proximal end region of the reinforcement layer can overlap with a distal end region of the proximal portion 366 such that a small overlap of hypotube and reinforcement exists near the transition between the proximal portion 366 and the elongate body 360.

The tubular portion of the catheter advancement element 300 can have an outer diameter that has at least one snug point. A difference between the outer diameter at the snug point and the inner diameter of the lumen at the distal end of the distal, catheter portion can be no more than about 0.015″ (0.381 mm), or can be no more than about 0.010″ (0.254 mm). The at least one snug point of this tubular portion can be a point along the length of the tubular portion. The at least one snug point of this tubular portion can have a length that is at least about 5 cm up to about 50 cm, including for example, at least about 6 cm, at least about 7 cm, at least about 8 cm, at least about 9 cm, at least about 10 cm, at least about 11 cm, or at least about 12 cm up to about 50 cm. This length need not be uniform such that the length need not be snug along its entire length. For example, the snug point region can include ridges, grooves, slits, or other surface features.

In other implementations, the entire catheter advancement element 300 can be a tubular element configured to receive a guidewire 500 through both the proximal portion 366 as well as the elongate body 360. For example, the proximal portion 366 can be a tubular element having a lumen that communicates with the lumen 368 extending through the elongate body 360. In some implementations, the proximal portion 366 can be a skived hypotube of stainless steel coated with PTFE having an outer diameter of 0.026″ (0.660 mm). In other implementations, the outer diameter can be between 0.024″ (0.610 mm) and 0.030″ (0.762 mm). In some implementations, such as an over-the-wire version, the proximal portion 366 can be a skived hypotube coupled to a proximal hub or luer 364. The proximal portion 366 can extend eccentric or concentric to the distal luminal portion 222. The proximal portion 366 can be a stainless steel hypotube. The proximal portion 366 can be a solid metal wire that is round or oval cross-sectional shape. The proximal portion 366 can be a flattened ribbon of wire having a rectangular cross-sectional shape. The ribbon of wire can be curved into a circular, oval, c-shape, or quarter circle, or other cross-sectional shape along an arc. The proximal portion 366 can have any of variety of cross-sectional shapes whether or not a lumen extends therethrough, including a circular, oval, C-shaped, D-shape, or other shape. In some implementations, the proximal portion 366 is a hypotube having a D-shape such that an inner-facing side is flat and an outer-facing side is rounded. The rounded side of the proximal portion 366 can be shaped to engage with a correspondingly rounded inner surface of the sheath 400. The hypotube can have a lubricious coating, such as PTFE or other lubricious polymer covering the hypotube. The hypotube can have an inner diameter of about 0.021″ (0.533 mm), an outer diameter of about 0.0275″ (0.699 mm), and an overall length of about 94 cm providing a working length for the catheter advancement element 300 that is about 143 cm. Including the proximal luer 364, the catheter advancement element 300 can have an overall length of about 149 cm. In some implementations, the hypotube can be a tapered part with a length of about 100 mm, starting proximal with a thickness of 0.3 mm and ending with a thickness of 0.10 mm to 0.15 mm. In still further implementations, the elongate body 360 can be a solid element coupled to the proximal portion 366 having no guidewire lumen.

The proximal portion 366 is shown in FIG. 9A as having a smaller outer diameter compared to the outer diameter of the elongate body 360. The proximal portion 366 need not step down in outer diameter and can also have the same outer diameter as the outer diameter as the elongate body 360. The proximal portion 366 can incorporate a hypotube or other stiffening element that is coated by one or more layers of polymer resulting in a proximal portion 366 having substantially the same outer diameter as the elongate body 360.

At least a portion of the solid elongate body 360, such as the elongate distal end region 346, can be formed of or embedded with or attached to a malleable material that skives down to a smaller dimension at a distal end. The distal end region 346 can be shaped to a desired angle or shape similar to how a guidewire may be used. The malleable length of the elongate body 360 can be at least about 1 cm, 3 cm, 5 cm, and up to about 10 cm, 15 cm, or longer. In some implementations, the malleable length can be about 1%, 2%, 5%, 10%, 20%, 25%, 50% or more of the total length of the elongate body 360. In some implementations, the catheter advancement element 300 can have a working length of about 140 cm to about 143 cm and the elongate body 360 can have an insert length of about 49 cm. The insert length can be the PEBAX portion of the elongate body 360 that is about 49.5 cm. As such, the malleable length of the elongate body 360 can be between about 0.5 cm to about 25 cm or more. The shape change can be a function of a user manually shaping the malleable length prior to insertion or the distal end region 346 can be pre-shaped at the time of manufacturing into a particular angle or curve. Alternatively, the shape change can be a reversible and actuatable shape change such that the distal end region 346 forms the shape upon activation by a user such that the distal end region 346 can be used in a straight format until a shape change is desired by the user. The catheter advancement element 300 can also include a forming mandrel extending through the lumen of the elongate body 360 such that a physician at the time of use can mold the distal end region 346 into a desired shape. As such, the moldable distal end region 346 can be incorporated onto an elongate body 360 that has a guidewire lumen.

The elongate body 360 can extend along the entire length of the catheter 200, including the distal luminal portion 222 and the proximal extension 230 or the elongate body 360 can incorporate the proximal portion 366 that aligns generally side-by-side with the proximal extension 230 of the catheter 200. The proximal portion 366 of the elongate body 360 can be positioned co-axial with or eccentric to the elongate body 360. The proximal portion 366 of the elongate body 360 can have a lumen extending through it. Alternatively, the portion 366 can be a solid rod or ribbon having no lumen.

Again with respect to FIGS. 9A-9B and 10A-10B, like the distal luminal portion 222 of the catheter 200, the elongate body 360 can have one or more radiopaque markers 344 along its length. The one or more markers 344 can vary in size, shape, and location. One or more markers 344 can be incorporated along one or more parts of the catheter advancement element 300, such as a tip-to-tip marker, a tip-to-taper marker, an RHV proximity marker, a Fluoro-saver marker, or other markers providing various information regarding the relative position of the catheter advancement element 300 and its components. The at least one radiopaque marker can identify the tapered end region of the elongate body 360. In some implementations and as best shown in FIGS. 10A-10B, a distal end region can have a first radiopaque marker 344a and a second radiopaque marker 344b can be located to indicate the border between the tapering of the distal end region 346 and the more proximal region of the elongate body 360 having a uniform or maximum outer diameter. This provides a user with information regarding an optimal extension of the distal end region 346 relative to the distal end of the luminal portion 222 to minimize the lip at this distal end of the luminal portion 222 for advancement through tortuous anatomy. In other implementations, for example where the distal end region 346 is not necessarily tapered, but instead has a change in overall flexibility along its length, the second radiopaque marker 344b can be located to indicate the region where the relative flexibilities of the elongate body 360 (or the distal end region 346 of the elongate body 360) and the distal end of the luminal portion 222 are substantially the same. The marker material may be a platinum/iridium band, a tungsten, platinum, or tantalum-impregnated polymer, a metallic coil or braid, or other radiopaque marker. The radiopaque marker(s) preferably do not impact the flexibility of the distal end region 346 and elongate body 360. In some implementations, the radiopaque markers are extruded PEBAX loaded with tungsten for radiopacity. In some implementations, the proximal marker band can be about 2.0 mm wide and the distal marker band can be about 2.5 mm wide to provide discernable information about the distal end region 346. In other implementations, the proximal marker is a different construction and/or material from the proximal marker. Additionally, the radiopaque marker bands 344a, 344b can be visible to a user without fluoroscopy, for example, prior to inserting the catheter system into the patient. The marker bands 344a, 344b can form a contrasting color visible to a user compared to a color of the polymer of the flexible elongate body, such as a black band relative to a white color of the polymer. The marker bands 344a, 344b can be useful in achieving a particular relative extension of the catheter advancement element 300 to the catheter 200 prior to insertion of the devices into an RHV.

The catheter 200 and catheter advancement element 300 (with or without a guidewire) can be advanced as a single unit through both turns of the carotid siphon. Both turns can be traversed in a single smooth pass or throw to a target in a cerebral vessel without the step-wise adjustment of their relative extensions and without relying on the conventional step-wise advancement technique with conventional microcatheters. The catheter 200 having the catheter advancement element 300 extending through it allows a user to advance them in unison in the same relative position from the first bend of the siphon through the second bend beyond the terminal cavernous carotid artery into the ACA and MCA. Importantly, the advancement of the two components can be performed in a single smooth movement through both bends without any change of hand position.

The catheter advancement element 300 can be juxtapositioned relative to the catheter 200 that provides an optimum relative extension between the two components for single smooth advancement. The catheter advancement element 300 can be positioned through the lumen of the catheter 200 such that its distal end region 346 extends just beyond a distal-most end 215 of the catheter 200. The distal end region 346 of the catheter advancement element 300 eliminates the stepped transition between the inner member and the outer catheter 200 thereby avoiding issues with catching on branching vessels within the region of the vasculature such that the catheter 200 may easily traverse the multiple angulated turns of the carotid siphon. The optimum relative extension, for example, can be the distal end region 346 of the elongate body 360 extending just distal to a distal-most end 215 of the catheter 200. A length of the distal end region 346 extending distal to the distal-most end 215 of the catheter 200 during advancement can be between 0.5 cm and about 4 cm. This juxtaposition can be a locked engagement with a mechanical element or simply by a user holding the two components together. The mechanical locking element can be a fixed or removable mechanical element 605 configured to connect to one or more of the catheter 200, the catheter advancement element 300, and the guidewire 500. The mechanical locking element 605 can be slidable along at least a length of the system components when coupled so that the mechanical attachment is adjustable. The mechanical locking element 605 can be a disposable feature or reusable for connecting to at least a portion of the shaft or a more proximal portion of the component, such as the luer or hub at a proximal end of the component. In some implementations, the mechanical locking element 605 can be clamped onto the catheter 200 and the catheter advancement element 300 in a desired relative position so that the two can be advanced together without the relative position being inadvertently changed. The relative position can be changed, if desired, while the mechanical locking element 605 is clamped onto the catheter 200 and the catheter advancement element 300. The mechanical locking element 605 can be additionally clamped onto a region of the guidewire 500 extending through the catheter advancement element 300 such that the relative position of all three components can be maintained during advancement until a relative sliding motion is desired. In still further implementations, the clamping position of the mechanical locking element 605 can be changed from engaging with a first combination of components (e.g., the catheter, catheter advancement element, and the guidewire) to a different combination of components (e.g., the catheter advancement element and the guidewire) depending on what phase of the method is being performed. In still further implementations, the guidewire 500 is held fixed relative to the catheter advancement element 300 via a rotating hemostatic valve coupled to the proximal hub 434 and the catheter advancement element 300 is held fixed to the catheter 200 by a separate mechanical locking element 605. Whether the relative position of the components is fixed by a mechanical element, a combination of mechanical elements, or by a user, the proximal portions 264 of each of the catheter 200 and the catheter advancement element 300 (and the guidewire 500, if present) are configured to be held at a single point by a user. For example, where the catheter and catheter advancement element are advanced and/or withdrawn manually, the single point can be between just a forefinger and thumb of the user.

The components can be advanced together with a guidewire, over a guidewire pre-positioned, or without any guidewire at all. In some implementations, the guidewire can be pre-assembled with the catheter advancement element 300 and catheter 200 such that the guidewire extends through a lumen of the catheter advancement element 300, which is loaded through a lumen of the catheter 200, all prior to insertion into the patient. The pre-assembled components can be simultaneously inserted into the sheath 400 and advanced together up through and past the turns of the carotid siphon. A guidewire may be located within the lumen 368 of the catheter advancement element 300 and parked proximal of the tapered distal end region 346 or proximal of the distal tip for potential use in the event the catheter advancement element without a guidewire does not reach the target location. For example, a distal tip of the guidewire 500 can be positioned about 5 cm to about 40 cm, or about 20 cm to about 30 cm proximal of the distal end region 346 of the catheter advancement element 300. At this location the guidewire does not interfere with the performance or function of the catheter advancement element. The guidewire can be positioned within the lumen of the catheter advancement element such that the distal end of the guidewire is within the catheter advancement element during the step of advancing the assembled system of devices together and is extendable from the catheter advancement element out the distal opening 326 when needed for navigation. In one example, a rescue guidewire is parked within the lumen of the catheter advancement element with a distal end of the guidewire about 0 cm to about 40 cm proximal or about 5 cm to about 35 cm proximal or about 7 cm to about 30 cm of the distal end of the catheter advancement element, preferably about 10 cm proximal of the distal end of the catheter advancement element. The guidewire at this parked position can provide additional support for the proximal portion of the system without affecting the flexibility and performance of the distal portion of the system.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter side-branches can be dangerous. Guidewires are typically 0.014″ to 0.018″ (0.356 mm-0.457 mm) in the neuroanatomy and will find and often traumatize dissection flaps or small branches that accommodate this size, which can lead to small bleeds or dissections and further occlusion. In a sensitive area like the brain these events can be catastrophic. The tendency of a guidewire to bunch and prolapse can also cause a leading edge to the guidewire that can be advanced on its own or as part of a tri-axial system to create dissection planes and traumatize small vessels.

In contrast, the catheter advancement element 300 described herein preferentially stays in the larger lumen of a conduit vessel. The catheter advancement element 300 delivers to the largest lumen within the anatomy even in light of the highly tortuous anatomy and curves being navigated. The catheter advancement element 300 can preferentially take the larger lumen at a bifurcation or dissection flap while also following the current of the greatest blood flow thereby maintaining the general direction and angulations of the parent vessel. In viewing the standard anatomy found in the cerebral vasculature, the Circle of Willis is fed by two vertebral and two carotid conduit arteries. As these four arteries are the access points to the cerebral anatomy—the course of the catheter advancement element 300 can be identified and has been validated in standard cerebral anatomy models.

In the anterior circulation where the conduit artery point of entry for cerebral endovascular procedures is the internal carotid artery (ICA), the catheter advancement element can guide the large-bore catheter to the M1 segment of the middle cerebral artery (MCA) bypassing the anterior communicating artery (ACA) and anterior temporal branch (ATB). The very flexible nature of the catheter advancement element 300 combined with the distal flexible nature of most cerebral catheters combine to allow delivery through severe tortuosity. Independent of the tortuous nature of the course of the arteries, the catheter advancement element 300 tends to navigate the turns and deliver to the largest offspring from a parent artery, for example, ICA to M1 segment of the MCA. The M2 level branching of the M1 can be variable, but is often seen to have two major M2 branches (superior and inferior) and, depending on the anatomy, which can vary significantly between patients, may be seen to bifurcate “equally” or “unequally.” If the caliber of the M2 branching is of similar size and angulation, the catheter advancement element 300 may take one of the two branches. If the target for catheter placement is not in a favorable angulation or size of artery, the catheter advancement element 300 may be curved (e.g., via shaping of a malleable distal tip) and directed or a guidewire may be used.

In some anatomies where the M2 bifurcation is “even” in size, a back-and-forth motion may aid in selecting one branch then the other while still avoid the need or use of a guidewire or a curved distal tip of the catheter advancement element. The back-and-forth motion can allow for the catheter advancement element to be directed into either branch of the M2. The catheter advancement element, even when initially straight, achieves some curvature that aids in directing it into a branch vessel. Thus, when an operator encounters an M2 bifurcation and there is a desire to cannulate either branch of an evenly divided bifurcation, selection of either branch is possible using the catheter advancement element without a guidewire.

Thus, main channels, such as the ICA, the middle cerebral artery and its tributaries in the anterior circulation will naturally be the pathway of preference for the described catheter advancement element and subsequence large-bore catheter delivery (via access from the ICA). A similar phenomenon can occur in the posterior circulation, which is accessed via the vertebral arteries arising from the subclavian arteries on the right and the left. The catheter advancement element will take the main channels in this circulation as well by traversing the vertebral arteries to the basilar artery and to the major tributaries of the basilar: the posterior cerebral artery and superior cerebellar arteries in the posterior circulation.

Navigation using the catheter advancement element can provide maximal deliverability with minimal vascular trauma. Catheters can cause “razoring” effects in a curved vessel because the blunt end of a large bore catheter can tend to take the greater curve in rounding a vessel when pushed by the operator. This blunt end can gouge or “razor” the greater curve with its sharp edge increasing the risk for dissection along an anatomic plane within the multilayered mid- or large-sized artery or vein (see, e.g., Catheter Cardiovasc. Interv. 2014 February; 83(2):211-20). The catheter advancement element can serve to minimize the edge of these catheters. Positioning the catheter advancement element within the lumen of the large-bore catheter such that the taper marker of the catheter advancement element is aligned optimally with the distal tip marker of the catheter minimizes the edge and thereby eliminates “razoring” as the large-bore catheter is advanced through turns of the vessel. This is particularly useful for the cerebral anatomy. Treatments distal to the carotid siphon, particularly distal to the ophthalmic artery takeoff from the greater curve of the severe tortuosity of the final turn of the carotid siphon “S-turn”, the “anterior genu” of the carotid siphon typically seen as part of the terminal internal carotid artery (ICA) can be improved using the access system described herein. The specifics of the catheter advancement element in proper alignment within the large bore catheter (the “tip-to-taper” position noted by the distal tip marker) relative to the taper marker of the catheter advancement element maximize the likelihood that razoring and hang-up on the ophthalmic artery are avoided during manual advancement of the catheter system. The taper marker of the catheter advancement element can be positioned at or past the take-off of the ophthalmic artery to minimize these deleterious effects and allows the large-bore catheter to pass the ophthalmic artery without incident. In a relatively straight segment, which is common after passing the siphon, the large-bore catheter can be advanced over the catheter advancement element, which serves still as a guiding element to the target. The transition between the catheter advancement element and the distal edge of the large-bore catheter is insignificant, especially compared to the step changes present with a typical microcatheter or guidewire, which do not prevent hang-ups on branches, such as the ophthalmic artery. The catheter advancement element allows for maneuvering of the large-bore catheter to distal sites without use of a microcatheter or guidewire.

The systems described herein can but need not incorporate a guidewire. And, if a guidewire is used, it need not be advanced independently (i.e., unsheathed) to the target treatment site. Thus, the systems described herein can incorporate relatively large bore catheters that are delivered without disturbing anatomy with a guidewire, reducing the risk for stroke and downstream effects from fragmentation of an occlusion, and having improved efficiency. Additionally, the systems described herein are single-operator systems allowing the operator to work at a single RHV and, in the case of spined components, can manipulate all the elements being used to navigate the anatomy with single-handed “pinches.” This can be referred to as “monopoint.”

Any flow diverter described herein may be used with any device delivery system including but not limited to those described here, and may be delivered via access catheters including but not limited to those described here.

### Methods of Use

The access catheter systems, flow diverters and delivery systems described herein can be used to access and treat intracranial and cerebral aneurysms. The access catheter systems provide monopoint manipulation at the base sheath for the various tools used in the method providing improved safety, ease of use, and single operator manipulations compared to conventional systems. These catheter systems provide easy and quick access to target sites even through tortuous anatomy to reach the target lesion. The flow diverters and delivery systems described herein provide improved, more accurate, and safer treatment of aneurysms. In addition, the flow diverters described here have potentially reduced complication rates due to the geometry of the device apposition against the wall.

A method for the treatment of cerebral or intracranial aneurysm is now described. The method can include a flow diverter and flow diverter delivery system advanced over a guidewire (or not) through an outer catheter extending through a base sheath. The catheter can be a conventional full-length catheter, but is preferably a catheter having a larger diameter distal luminal portion 222 coupled to a smaller diameter proximal control element 230 as shown in FIGS. 9A-9B so that monopoint manipulation at the base sheath hub is possible. The base sheath 400 can be introduced into a blood vessel (e.g., femoral artery) and advanced to the level of at least the common carotid artery towards an intracranial or cerebral vessel having a segment with an aneurysm. An outer catheter 200 is advanced through the hub (e.g., an RHV 434) on the base sheath 400 until the distal end of the catheter 200 exits the distal opening 408 of the base sheath 400 (see FIG. 11A). The catheter 200 can be advanced into the high ICA. The outer catheter 200 can be part of a catheter system including an inner catheter 300 having a tapered end region 346 that extends distal to the distal end of the outer catheter 200. The outer catheter 200 can be navigated through the carotid siphon CS towards the aneurysm A aided by the inner catheter 300. The outer catheter 200 and inner catheter 300 can be advanced until at least a portion of the tapered end region 346 of the inner catheter 300 is positioned across the target aneurysm as illustrated in FIG. 11A. Alternatively, a guidewire 500 can be advanced through the hub on the base sheath 400 and advanced until the guidewire 500 is positioned across the aneurysm A while the outer catheter 200 remains parked at a location between the distal end of the base sheath 400 and the aneurysm A (e.g., at or near the carotid siphon CS).

The distal end region of the outer catheter 200 can be advanced over the inner catheter 300 and positioned across the aneurysm A. The inner catheter 300 can be withdrawn from the outer catheter 200 and the outer catheter 200 maintained in position across the aneurysm (see FIG. 11B). The outer catheter 200 can have an ID of between 2.0 mm and 3.0 mm that is configured to receive a flow diverter 700 mounted within a flow diverter delivery system 800. The flow diverter delivery system 800 and flow diverter 700 can be advanced (e.g., through the hub of the outer catheter 200 or the hub of the base sheath 400 and into the distal tubular portion of the catheter 200 if the catheter 200 is a partial length catheter) to the distal end region of the outer catheter 200. The outer catheter 200 can be withdrawn to expose the flow diverter delivery system 800 while the flow diverter delivery system 800 is maintained across the aneurysm A (see FIG. 11C). The flow diverter 700 of the flow diverter delivery system 800 can then be deployed across the aneurysm A (see FIG. 11D).

The flow diverter delivery system 800 can include an inner core member 820 and an outer restraining sleeve 810. The flow diverter 700 can be mounted on the inner core member 820 and constrained by the outer restraining sleeve 810 during delivery. The flow diverter 700 constrained by the outer restraining sleeve 810 can be deliverable through a delivery catheter having an inner diameter that is between 2.0 mm and 3.0 mm. Deployment of the flow diverter 700 across the aneurysm A can be achieved, for example, in reference to FIG. 11D, by retracting the outer restraining sleeve 810 of delivery system 800 to expose the flow diverter 700 while the inner core member 820 remains in place distal to the aneurysm A.

The flow diverter 700 can be any of those described previously. For example. The flow diverter can be a laser-cut expandable metal tube. The flow diverter can be formed of first and second expandable tubes where each is a laser cut metal tube. The first expandable tube can be a laser cut metal tube and the second expandable tube can be a braided tube. Alternatively, the first expandable tube can be a laser cut metal tube and the second expandable tube can be a polymer sleeve. The flow diverter can have a compound construction. The compound construction can include two end sections constructed from laser-cut tube and a middle section that is a braid.

### Materials

One or more components of the catheters, delivery systems, and flow diverters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes, such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable cut-tube or flat metal material includes Nitinol, Layered tube with Nitinol on outside and inner core of radiopaque material, such as tantalum, platinum, iridium, gold, alloy etc. Additionally, material could be cobolt, cobolt alloy, or stainless steel.

Some examples of suitable metals and metal alloys include stainless steel, such as 304V, 304L, and 316LV stainless steel; mild steel; nickel-titanium alloy, such as linear-elastic and/or super-elastic Nitinol; other nickel alloys, such as nickel-chromium-molybdenum alloys (e.g., UNS: N06625, such as INCONEL® 625, UNS: N06022, such as HASTELLOY® C-22®, UNS: N10276, such as HASTELLOY® C276®, other HASTELLOY® alloys, and the like), nickel-copper alloys (e.g., UNS: N04400, such as MONEL® 400, NICKELVAC® 400, NICORROS® 400, and the like), nickel-cobalt-chromium-molybdenum alloys (e.g., UNS: R30035, such as MP35-N® and the like), nickel-molybdenum alloys (e.g., UNS: N10665, such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003, such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers, such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking, such as metals including stainless steel, Nitinol, Nitinol braid, helical ribbon, helical wire, cut stainless steel, or the like, or stiff polymers, such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials, such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA) or heparin encased in alginate.

Implementations describe catheters and delivery systems and methods to deliver catheters to target anatomies. However, while some implementations are described with specific regard to delivering catheters to a target vessel of a neurovascular anatomy, such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The components of the systems disclosed herein may be packaged together in a single package or separately. The finished package would be sterilized using sterilization methods such as Ethylene oxide or radiation and labeled and boxed. Instructions for use may also be provided in-box or through an internet link printed on the label.

What is claimed is:

1. A flow diverter comprising:
a self-expanding tubular member comprising a plurality of expandable cells, each of the expandable cells comprising interconnected struts and bridges,
wherein the tubular member has a constrained configuration having a first outer diameter of at least 1.0 mm sized for delivery using a flow diverter delivery system and an expanded configuration having a second outer diameter larger than the first outer diameter, and
wherein the tubular member has a proximal end zone, a distal end zone, and a middle zone located between the proximal end zone and the distal end zone, wherein at least the middle zone of the tubular member is laser-cut to have a material coverage of at least 25% when the tubular member is in the expanded configuration.

2. The flow diverter of claim 1, wherein the interconnected struts and bridges of each expandable cell comprises two pairs of struts each strut of the two pairs of struts having an outer edge, wherein the outer edge of a first strut of a first pair is interconnected to an outer edge of a second strut of the first pair by one of the bridges.

3. The flow diverter of claim 2, wherein the first strut of the first pair of struts connects at a central bend to a first strut of a second pair of struts, the second strut of the first pair connects at a central bend to a second strut of the second pair, wherein a circumferential height from the central bend of the first pair to the outer edge of the first pair is Y and an axial distance from the central bend of the first pair to the outer edge of the first pair is X, and wherein a diagonal of a rectangle defined by X and Y is equal to a length of the first strut.

4. The flow diverter of claim 3, wherein a ratio of the length of the first strut to the circumferential height of the first strut is between 1 and 5.

5. The flow diverter of claim 2, wherein each of the pairs of struts is arranged parallel to one another and spaced an axial distance away from one another thereby defining a V-shaped opening of the expandable cell.

6. The flow diverter of claim 5, wherein the two pairs of struts are interconnected to form a peak on a first end of the expandable cell and a corresponding valley on a second end of the expandable cell.

7. The flow diverter of claim 6, wherein the plurality of expandable cells are arranged in circumferential rings and each peak in a circumferential ring of expandable cells is aligned circumferentially with each peak of an adjacent circumferential ring of expandable cells.

8. The flow diverter of claim 6, wherein a bridge connects the peak of the expandable cell of a first circumferential ring to a valley of an expandable cell of an adjacent second circumferential ring.

9. The flow diverter of claim 1, wherein the middle zone has properties different from one or both of the proximal end zone and distal end zone.

10. The flow diverter of claim 1, wherein the middle zone has greater material coverage than one or both of the proximal end zone and the distal end zone.

11. The flow diverter of claim 1, wherein one or both of the proximal end zone and the distal end zone is laser-cut to have a material coverage that is less than the material coverage of the middle zone.

12. The flow diverter of claim 1, wherein the material coverage of the middle zone is between 25%-35% when the tubular member is in the expanded configuration and the proximal and distal end zones have a material coverage less than the material coverage of the middle zone.

13. The flow diverter of claim 1, wherein at least one of the proximal end zone, the middle zone, and the distal end zone comprises at least one radiopaque marker.

14. The flow diverter of claim 1, wherein a length of the flow diverter in the constrained configuration is less than 1% different from a length of the flow diverter in the expanded configuration.

15. The flow diverter of claim 1, wherein a length of the flow diverter in the constrained configuration is less than about 5% different from a length of the flow diverter in the expanded configuration.

16. The flow diverter of claim 1, wherein a length of the flow diverter in the constrained configuration is less than about 10% different from a length of the flow diverter in the expanded configuration.

17. The flow diverter of claim 1, wherein the first outer diameter is between 1.5 mm and 2.5 mm and wherein the second outer diameter is between 2.0 mm and 6.0 mm.

18. The flow diverter of claim 1, wherein a length of the flow diverter in the constrained configuration is between 10 mm and 35 mm.

19. The flow diverter of claim 1, wherein the plurality of expandable cells of the tubular member is arranged into between 10 and 50 circumferential rings.

20. The flow diverter of claim 1, wherein a pitch of the middle zone is between about 0.25 mm-0.40 mm, the pitch corresponding to a length of a bridge of an expandable cell of the middle zone.

21. The flow diverter of claim 20, wherein a pitch of one or both of the proximal end zone and distal end zone is about 0.45 mm-0.75 mm, the pitch corresponding to a length of a bridge of an expandable cell of the proximal end zone or distal end zone.

22. The flow diverter of claim 1, wherein the plurality of expandable cells form rows extending between proximal and distal ends of the tubular member parallel with a longitudinal axis of the tubular member, the rows of the expandable cells aligned peak-to-valley.

23. The flow diverter of claim 22, wherein the tubular member comprises between 4 and 10 rows.

24. The flow diverter of claim 22, wherein at least the distal end zone comprises a rail formed of bridges interconnecting the plurality of expandable cells within a row.

25. The flow diverter of claim 24, wherein the rail enables re-sheathing of the distal end zone in a delivery system after at least partial deployment of the distal end zone.

26. The flow diverter of claim 1, wherein one or both of the proximal end zone and the distal end zone comprises a braided or woven construction.

27. The flow diverter of claim 1, wherein the interconnected struts are connected by hinges in a plurality of V-shapes.

28. The flow diverter of claim 27, wherein a line connecting radially adjacent hinges passes through at least 4 cells in the middle zone.

29. The flow diverter of claim 28, wherein the line connecting radially adjacent hinges in the proximal and distal zones passes through fewer cells than in the middle zone.

30. The flow diverter of claim 1, wherein bridges located in the middle zone are shorter than bridges located in the distal end zone and proximal end zone, and wherein the struts in the middle zone, distal end zone, and proximal end zone are substantially the same in length and configuration.

31. The flow diverter of claim 1, wherein the bridges lie parallel to a flow diverter central axis.

32. A method of treating intracranial or cerebral aneurysm, the method comprising:
advancing a catheter system through a base sheath towards an intracranial or cerebral vessel having a segment with an aneurysm, the catheter system comprising:
an inner catheter having a tubular elongate body with a single lumen and a flexible, distal tapered end region; and
an outer catheter having a catheter lumen and a distal end;
positioning the tapered end region of the inner catheter distal to the distal end of the outer catheter;
crossing the segment of vessel with the aneurysm with at least a portion of the tapered end region of the inner catheter;
advancing the outer catheter over the inner catheter and positioning a distal end region of the outer catheter across the lesion;
withdrawing the inner catheter from the catheter lumen and maintaining the outer catheter in place across the aneurysm;
advancing a flow diverter delivery system comprising a flow diverter through the catheter lumen to the distal end region of the outer catheter;
withdrawing the outer catheter while maintaining the flow diverter delivery system in place; and
deploying the flow diverter across the segment with the aneurysm.

33. A method of performing a medical procedure at a treatment site in a brain of a patient, the method comprising:
positioning a system of devices into an advancement configuration, the system of devices comprising:
a catheter having a catheter lumen, an inner diameter, and a distal end; and
an inner member sized and shaped to slide within the catheter lumen, wherein the inner member defines a single lumen and has a distal portion,
wherein the distal portion has a first outer diameter that tapers distally to a second outer diameter that is smaller than the first outer diameter, and
wherein the inner member transitions in flexibility from a proximal end of the inner member to a distal end of the inner member, the distal end of the inner member being more flexible than the distal end of the catheter, and
wherein, when positioned in an advancement configuration, the inner member extends coaxially through the catheter lumen until the distal portion of the inner member is positioned distal to the distal end of the catheter;
advancing the catheter and the flexible inner member to a target location to an access point of entry while the system of devices is positioned in the advancement configuration;
positioning the catheter at the treatment site, the treatment site comprising an aneurysm;
removing the inner member from the patient; and
treating the aneurysm through the catheter.

34. The method of claim 33, wherein the step of treating comprises delivering a flow diverter to the aneurysm through the catheter.

35. A flow diverter delivery system comprising:
a flow diverter having a tubular structure and configured to treat an aneurysm in an intracranial vessel, the flow diverter comprising a constrained configuration having a first outer diameter and an expanded configuration having a second outer diameter;
an inner core member comprising:
an elongate shaft comprising a recessed region near a distal end region of the elongate shaft, the recessed region sized to receive the tubular structure of the flow diverter when the flow diverter is in the constrained configuration; and
an atraumatic distal tip region located distal to the recessed region, the distal tip region having a taper from a first outer diameter of the elongate shaft to a second outer diameter of the elongate shaft, wherein the first outer diameter of the elongate shaft is larger than an outer diameter of the recessed region; and
an outer restraining sleeve having an inner diameter sized to receive the inner core member and the flow diverter in the constrained configuration, wherein the outer restraining sleeve is retractable at least a distance to deploy the flow diverter.

36. The flow diverter delivery system of claim 35, wherein the inner diameter of the restraining sleeve is size-matched to the first outer diameter of the elongate shaft to reduce an annular space at a leading end of the flow diverter delivery system.

37. The flow diverter delivery system of claim 35, wherein the distal tip region comprises at least one radiopaque marker at a distal end.

38. The flow diverter delivery system of claim 37, wherein the distal tip region comprises a second radiopaque marker, wherein the second radiopaque marker is positioned to identify the taper.

39. A flow diverter comprising:
a self-expanding tubular member having a proximal end, a distal end, and a longitudinal axis, the tubular member having a constrained configuration with a first outer diameter sized for delivery and an expanded configuration having a second outer diameter larger than the first outer diameter,
wherein the tubular member comprises a plurality of expandable cells, each cell comprising interconnected struts and bridges arranged in circumferential rings, the circumferential rings forming rows of the expandable cells extending between the proximal and distal ends of the tubular member parallel with the longitudinal axis, the rows of expandable cells nested peak-to-valley,
wherein the tubular member has a proximal end zone near the proximal end of the tubular member, a distal end zone near the distal end of the tubular member, and a middle zone located between the proximal end zone and the distal end zone,
wherein at least the distal end zone comprises at least one rail formed of bridges interconnecting each circumferential ring of expandable cells within a single row.

40. The flow diverter of claim 39, wherein the at least one rail enables re-sheathing of the distal end zone in a delivery system after at least partial deployment of the distal end zone from the delivery system.

41. The flow diverter of claim 39, wherein at least the middle zone of the tubular member is laser-cut to have a material coverage of at least 25% when the tubular member is in the expanded configuration.

42. A flow diverter configured to expand from a constrained state to an expanded state, the flow diverter comprising:
a first tube of superelastic material formed of a plurality of cells having a first material coverage; and
a second tube of superelastic material formed of a plurality of cells having a second material coverage,
wherein the second tube is positioned inside of the first tube so that an overlap of the plurality of expandable cells of the first tube and the plurality of expandable cells of the second tube forms a third material coverage that is greater than the first material coverage and the second material coverage when the flow diverter is in the expanded state.

43. The flow diverter of claim 42, wherein the second tube is locked in position inside the first tube by a feature in a cut pattern of at least one of the first tube and the second tube.

44. The flow diverter of claim 43, wherein the feature comprises a slot in the first or the second tube and tab configured to protrude into the slot to lock the first and second tubes together.

45. The flow diverter of claim 43, wherein the feature comprises a hole in the first or the second tube and a malleable disk configured to insert within the hole to lock the first and second tubes together.

46. The flow diverter of claim 42, wherein at least one of the first tube and the second tube is non-braided and laser-cut.
